(19) United States
(12) Patent Application Publication
(10) Pub. No.: US 2003/0166213 A1
(43) Pub. Date: Sep. 4, 2003

(54) Methods for identifying compounds that modulate disorders related to nitric oxide/ cGMP-dependent protein kinase signaling

(76) Inventors: Ralph J. Greenspan, Coronado, CA (US); Paul J. Shaw, San Diego, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/738,630

(22) Filed: Dec. 15, 2000

(51) Int. Cl. A61K 49/00; C07H 21/04; C12Q 1/68; C12N 9/12

(52) U.S. Cl. 435/194; 424/9.2; 435/6; 536/23.2

(57) ABSTRACT

The invention provides a method of identifying a compound that modulates a disease associated with a nitric oxide/cGMP-dependent protein kinase network in a mammal. The method consists of (a) administering a test compound to an invertebrate; and (b) measuring a foraging behavior of the invertebrate, wherein a compound that modulates the foraging behavior of the invertebrate is characterized as a compound that modulates a disease associated with a nitric oxide/cGMP-dependent protein kinase network in a mammal.

### BACKGROUND OF THE INVENTION

Attention Deficit/Hyperactivity Disorder (ADHD) affects approximately 4% to 6% of the U.S. population. The most common core features include excessive and pervasive forms of distract ability (poor sustained attention to tasks), impulsivity (impaired impulse control and delay of gratification) and hyperactivity (excessive activity and physical restlessness). ADHD is a clinical diagnosis which can be broken down into subtypes known as combined type, predominantly inattentive type, and predominantly hyperactive-impulsive type. Although individuals lacking the hyperactivity component of ADHD are often referred to as having ADD, or attention deficit disorder, the diagnosis of ADHD is known in the medical arts to encompass all forms described above.

ADHD is commonly diagnosed in children and usually persists throughout a person's lifetime such that approximately one-half to two-thirds of children with ADHD will continue to have significant problems with ADHD symptoms and behaviors as adults. For both children and adults, ADHD can have significant impact on their lives on the job, within the family, and in social relationships. In addition to symptoms of ADHD, it is common for people having the disorder to present with co-morbid conditions such as anxiety disorders, substance abuse, or learning disabilities.

Currently available drugs to treat ADHD are stimulant medications such as ritalin (methylphenidate), dexadrine or adderall. These stimulants can have a variety of undesirable side effects such as stomach upset, cramps, loss of appetite, diarrhea, headache, nervousness, dizziness, sleep problems, irritability or restlessness, and in the extreme: seizures or convulsions, blurred vision, heart problems and unusual bleeding or bruising.

About 50 million adults in the U.S. have hypertension, a condition to which 20 to 50% of all natural deaths can be traced. Untreated hypertension can damage the kidneys and lead to stroke, heart attack or heart failure. Currently available treatments can also cause health problems. Hypertension is usually treated with diuretics, beta blockers or calcium blockers, drugs which can cause fatigue, weakness, headache, joint and stomach aches, nausea, impotence or urinary tract problems. In some cases the potential benefits of antihypertensives may not outweigh their negative effects on quality of life. In this regard, increasing numbers of antihypertensive medications are associated with lower reported general health status.

Clearly there is a need to identify drugs that alleviate ADHD and hypertension without undesirable side effects. At present there do not exist good methods of screening for such drugs. The present invention satisfies this need and provides related advantages as well.

### SUMMARY OF THE INVENTION

The invention provides a method of identifying a compound that modulates a disease associated with a nitric oxide/cGMP-dependent protein kinase network in a mammal. The method consists of (a) administering a test compound to an invertebrate; and (b) measuring a foraging behavior of the invertebrate, wherein a compound that modulates the foraging behavior of the invertebrate is characterized as a compound that modulates a disease associated with a nitric oxide/cGMP-dependent protein kinase network in a mammal.

The invention also provides a method of identifying a polynucleotide that correlates with a disease associated with nitric oxide/cGMP-dependent protein kinase network in a mammal. The method consists of (a) obtaining a first and a second strain of an invertebrate; (b) subjecting the first and second invertebrate strains to conditions in which the first strain exhibits a foraging behavior different than a foraging behavior exhibited by the second strain; (c) measuring an expression level for one or more polynucleotides in the first and second strains, and (d) identifying one or more polynucleotides that are differentially expressed in the first strain relative to the second strain, wherein a mammalian polynucleotide comprising substantially the same nucleic acid sequence as the one or more differentially expressed polynucleotides correlates with ADHD in a mammal.

### BRIEF DESCRIPTION OF THE DRAWINGS

FIG. 1 shows a schematic of the foraging maze used in measuring foraging scores of Drosophila.

FIG. 2 shows the effects of transferring flies to new tubes on rest. The amount of rest in minutes is plotted for each hour of a 24 hour day. Time 0 on the graph is at “lights-on” as described in Example IV. During baseline (squares) flies show a typical amount and distribution of rest. After being physically transferred to new tubes (circles) all flies obtained similar amounts of rest to baseline indicating that the transfer process did not alter the sleep-wake cycle.

FIG. 3 shows the effects of methylphenidate on rest in flies. The amount of rest in minutes is plotted for each hour of a 24 hour day. Time 0 on the graph is at “lights-on” as described in Example IV. During baseline (squares) flies show a typical amount and distribution of rest. After being physically transferred to new tubes containing 0.5 mg/ml methylphenidate (circles) rest was significantly reduced compared to baseline indicating that the methylphenidate can increase waking and reduce sleep in an invertebrate.

### DETAILED DESCRIPTION OF THE INVENTION

The methods and compounds disclosed herein are based on the discovery that foraging behavior in an invertebrate can be changed by altering molecular components of the nitric oxide/cGMP-dependent protein kinase network. In this regard, it is demonstrated herein that administration of a compound used for treating ADHD in humans can be effective in changing foraging behavior of invertebrates. Accordingly, the present invention provides methods of rapidly and efficiently identifying compounds that modulate attention deficit hyperactivity disorder (ADHD) in a mammal. The invention also provides methods for rapidly and efficiently identifying compounds that modulate other diseases involving the nitric oxide/cGMP-dependent protein kinase network in a mammal including, for example, hypertension.

A method of identifying a compound that modulates ADHD consists of administering a test compound to an invertebrate and measuring a foraging behavior of the invertebrate. A candidate compound that modulates the foraging behavior of the invertebrate is characterized as a compound that modulates ADHD. The method can also be used to identify compounds that modulate other diseases involving the nitric oxide/cGMP-dependent protein kinase network in a mammal including, for example, hypertension. A compound identified by the methods of the invention can be used to treat an individual suffering from ADHD or hypertension.

According to the methods of the invention, changes in expression of one or more representative genes in a network of genes can be associated with changes in foraging behavior of an invertebrate to identify compounds that modulate the nitric oxide/cGMP-dependent protein kinase network. Representative genes can include one gene, a subset of genes, or a set of all the genes whose expression changes upon modulation of ADHD or hypertension in a mammal. Therefore, the present invention also provides mammalian genes that modulate ADHD or hypertension. A compound identified by the method of the invention can act to modulate the activity or expression of a mammalian gene of the invention.

Until now, there has been no indication that the relative genetic simplicity of invertebrates compared to mammals provides networks of genes controlling foraging behavior in invertebrates that are similar to those affecting behavioral disorders such as ADHD in mammals or cardiopulmonary disorders such as hypertension. In fact, invertebrates have been shown to have dissimilar pharmacological responses compared to mammals within the same class of neuronal receptors. Thus, the use of invertebrates to test compounds affecting behavioral and cardiopulmonary disorders in mammals have not been pursued previous to this disclosure.

The methods of the invention provide a means to identify compounds that modulate ADHD, hypertension, or other diseases involving the nitric oxide/cGMP-dependent protein kinase network by screening foraging behavior of invertebrates which is a natural genetically encoded network. Thus, the methods provide for screening compounds in the context of a natural system which represents the mammalian nitric oxide/cGMP-dependent protein kinase network more closely than any in vitro assay could. Additionally, the degree to which compounds modulate a mammalian system can be identified by the methods of the invention because strains of invertebrates that have naturally evolved different foraging behaviors and degrees of response can be used with the methods of the invention.

As used herein, the term “compound” refers to an inorganic or organic molecule such as a drug; a peptide, or a variant or modified peptide or a peptide-like molecule such as a peptidomimetic or peptoid; or a polypeptide such as an antibody, a growth factor, or cytokine, or a fragment thereof such as an Fv, Fd or Fab fragment of an antibody, which contains a binding domain; or a polynucleotide or chemically modified polynucleotide such as an antisense polynucleotide; or a carbohydrate or lipid.

As used herein, the term “test compound” refers to an inorganic or organic molecule that is administered to an invertebrate for the purpose of determining its effects on an invertebrate foraging behavior. A test compound can be administered as a pure preparation or as a mixture with one or more other molecule. For example, a test compound can be combined with, or dissolved in, an agent that facilitates uptake of the compound by the invertebrate, such as an organic solvent, for example, DMSO or ethanol; or an aqueous solvent, for example, water or a buffered aqueous solution; or food.

As used herein the term “administering” when used in reference to a compound and an invertebrate refers to delivering the compound to the invertebrate in a manner allowing internalization of the test compound. A compound can be delivered, for example, by ingestion, inhalation, topically, or injection. Examples of delivery by ingestion include feeding a compound to invertebrates or adding a compound to an invertebrate's food. Topical delivery includes, for example, exposing an invertebrate to an aerosol preparation of a compound or a liquid preparation of a compound such that the compound contacts-the exterior or interior membranes.

As used herein, the term “ADHD” refers to attention deficit hyperactivity disorder which is a diagnosis applied to an individual who displays symptoms of excessive distractability, impulsivity, or hyperactivity. Attention deficit hyperactivity disorder includes attention deficit disorder (ADD). Attention deficit hyperactivity disorder can be subdivided into clinical subtypes including, for example, predominantly inattentive type, predominantly hyperactive-impulsive type or combined type. An individual with attention deficit hyperactivity disorder can display a reduction in distractability, impulsivity, or hyperactivity when administered stimulants including, for example, methylphenidate (Ritalin), amphetamines such as dexedrine and adderall, or pemoline (Cylert).

As used herein, the term “hypertension” refers to abnormally high blood pressure. High blood pressure is a relative measure that depends upon a statistical estimate of the distribution of systolic and diastolic blood pressures in the general population. As blood pressure is affected by factors such as age and gender, one skilled in the art can recognize high blood pressure by comparison to a cohort of matched individuals. Abnormally high blood pressure can be recognized as deviations from normal systolic blood pressure which include, for example, 80 mm Hg for an infant, 130 mm Hg for a 20 year old male and 140 mm Hg for a 40 year old male. Symptoms of hypertension can include, for example, drowsiness, confusion, headache, nausea, and loss of vision. Other symptoms can include, for example, increased risk for arteriosclerosis, angina pectoris, sudden death, stroke, dissecting aortic aneurysm, intra cerebral hemorrhage, rupture of the myocardial wall or artherothrombotic occlusion of the abdominal aorta.

As used herein the term “modulates” refers to an increase, decrease or alteration. The term can be used to indicate an increase, decrease or alteration of a phenotype or behavior. For example, an alteration of a foraging behavior can be a loss or gain of a behavior. An increased foraging behavior can be an increased duration of search, frequency of searching, energy expended in searching, distance of search, rate of search, area of search, or diligence of search. Accordingly, a decreased foraging behavior refers to, for example, a decreased duration of search, frequency of searching, energy expended in searching, distance of search, rate of search, area of search, or diligence of search. An alteration of phenotype can be, for example, a loss or gain of a characteristic associated with a particular genotype. Examples of increased phenotype include increased foraging behavior in a Rover or decreased foraging behavior in a sitter. A decreased phenotype can include, for example, decreased foraging behavior in a Rover or increased foraging behavior in a sitter.

A condition can be modulated by altering severity, extent, intensity, magnitude, duration or frequency of the condition. Modulating a condition can also include, for example, altering severity, extent, intensity, magnitude, duration or frequency of a symptom of the condition. Modulation of an expression level of a polynucleotide includes, for example, an increase in an amount of polynucleotide or polypeptide produced from the polynucleotide or decrease in an amount of polynucleotide or polypeptide produced from the polynucleotide. Modulation of an expression level of a polypeptide includes, for example, an increase in an amount of polypeptide produced from a polynucleotide or decrease in an amount of polypeptide produced from a polynucleotide. Modulation of an activity of a polypeptide includes, for example, increasing catalytic rate, decreasing catalytic rate, changing binding specificity, increasing binding rate or decreasing binding rate.

As used herein the term “invertebrate” refers to an animal that lacks a backbone. Invertebrates are understood to refer to members of the division invertebrata as understood in the art. An invertebrate can be a fly including, for example, fruit flies, sand flies, mayflies, blowflies, flesh flies, face flies, houseflies, screw worm-flies, stable flies, mosquitos, northern cattle grub, and the like As disclosed herein, is an example of an invertebrate. Fruit flies include Drosophila species such as D. melanogaster, D. simulans, D. virilis, D. pseudoobscura D. funebris, D. immigrans, D. repleta, D. affinis, D. saltans, D. sulphurigaster albostrigata and D. nasuta albomicans. Other invertebrate insects include, for example, cockroaches, honeybees, wasps, termites, grasshoppers, moths, butterflies, fleas, lice, boll weevils, beetles, Apis mellifera, A. florea, A. cerana, Tenebrio molitor, Bombus terrestris, B. lapidarius, and members of Hydrocorisae. Arthropods are also invertebrates including, for example, scorpions, spiders, mites, crustaceans, centipedes and millipedes. Other invertebrates include, for example, flatworms, nematodes (e.g. C. elegans), mollusks (e.g. Aplysia or Hermissenda), echinoderms and annelids will exhibit foraging behavior and express polynucleotides associated with foraging behavior, and can be used in the methods of the invention.

As used herein, the term “reference invertebrate” refers to a member of the division invertebrate or population of such members for which a polynucleotide expression level has been measured such that a polynucleotide expression level for another invertebrate can be compared. A polynucleotide expression level for a population of invertebrates can be an average, mean or median value derived from polynucleotide expression levels of individual invertebrates in the population. A reference invertebrate can be the same or different species as the invertebrate with which expression levels will be compared.

As used herein the term “adult,” when used in reference to Drosophila melanogaster refers to an individual that has passed the pupal stage. An adult can be distinguished from individuals in other developmental stages according to the presence of wings and fertility to reproduce.

As used herein the term “larva,” when used in reference to Drosophila melanogaster refers to an individual that has hatched from an egg and has not yet formed an immobile pupa. The larval stages include, for example, first, second and third instars.

As used herein the term “mammal” refers to a vertebrate animal distinguishable from other vertebrate animals, for example, by self regulating body temperature, hair or, in the female, mammee. Examples of mammals include a human, dog, cat, or horse.

As used herein the term “foraging behavior” refers to the actions of an individual or population of individuals in the presence of food or in a fed state. Actions included in the term can be for example, duration of search, frequency of searching, energy expended searching, distance of search, direction of search, rate of search, area of search, efficiency of search or diligence of search.

As used herein the term “different,” when used in reference to foraging behavior of individuals, refers to a measured value of a foraging behavior of a first invertebrate or a mean value of foraging behaviors measured for individuals in a population that is different from a measured value of a foraging behavior of a second invertebrate or a mean value of foraging behaviors measured for individuals in a second population if a pairwise t-test of two scores is significantly different at the 0.05 level, or if multiple pairwise comparisons between individuals are significantly different after applying a correction for experiment wise-error. A significantly different score refers to a score that is different by a statistically meaningful amount. Two foraging behaviors are also considered different if a first measured value for a foraging behavior is not within a desired region of the probability distribution of a second measured value for a foraging behavior. For example, a first measured value for a foraging behavior can be different if it is not within the 80% probable region of a probability distribution of the second measured value of a foraging behavior, or within the 85%, 90%, 95% or 98% probable region of the distribution of the second measured value of a foraging behavior. Correspondingly, measured values of a foraging behavior considered to be substantially the same are values that do not differ by a more than a desired standard deviation or are within a desired probable region of a probability distribution. Methods for the determination of mean, standard deviation and characteristics of normal distributions are known in the art as demonstrated by texts such as Biostatistical Analysis, 4th ed., Zar, Prentice-Hall Inc. (1999).

As used herein the term “sitter” refers to an individual Drosophila melanogaster that is homozygous for for^{S} alleles. The term “Rover” as used herein refers to an individual Drosophila melanogaster that contains a for^{R} allele. Rover/sitter is a polymorphism in Drosophila melanogaster. The phenotype of a Rover and sitter can be distinguished, for example, according to the distance traveled during foraging such that a Rover travels a longer pathlength when foraging than does a sitter. In the absence of food, or in a starved condition, Rovers and sitters show similar mobility.

As used herein the term “expression level,” when used in reference to a polynucleotide, refers to a quantity of translation or transcription product produced from a polynucleotide in a given condition. A polynucleotide expression level can be, for example, an amount of RNA translated from a DNA, an amount of polypeptide translated from an RNA or an amount of polypeptide produced from transcription and translation of a DNA. Accordingly, increased expression refers to a larger quantity of transcription or translation product produced in a time period or a faster rate of producing transcription or translation products. Correspondingly, decreased expression refers to a smaller quantity of transcription or translation product produced in a time period or a slower rate of producing transcription or translation products.

As used herein the term “differentially expressed” refers to dissimilar quantities of translation or transcription product produced from a polynucleotide in a given condition. Dissimilar quantities can be identified by the variance relative to a reference quantity. A reference quantity can be, for example, the variability of expression levels between invertebrates having, for example, similar genetic makeup, age, gender, developmental conditions, or the like. In such a situation, a different level can be a difference that is greater than the mean difference observed between expression levels of the invertebrates, or greater than the largest expression level difference observed between selected polynucleotides of the invertebrates. Different levels can also be based on the composite variability of polynucleotide expression levels between two or more strains. For example, the mean or median difference between polynucleotide expression levels can be determined between a large number of different strains. Any difference in expression that is greater than the mean or median difference can be considered differentially expressed. Other reference levels defining a significant difference can be determined by one of skill in the art according to the desired comparison between two or more invertebrates.

Differential expression can also be determined for invertebrates of the same strain that have been subjected to conditions in which a first group of members of a strain exhibit a foraging behavior different from the foraging behavior of a second group of members of the strain. This can be carried out, for example, by administration of a compound, presence of light, time of day, and the like. Differential expression is then determined by measuring expression levels in the two groups and identifying polynucleotides expressed at significantly different levels.

As used herein, a “strain” refers to a population of organisms of a species having at least one similar phenotype including, for example, a foraging phenotype. This population of organisms can have either identical or a somewhat heterogeneous genetic makeup, although heterogeneous populations typically contain individuals that are homozygous for one or more chromosomes. For example, a population of organisms having a similar phenotype can be a population of organisms of a species sharing a similar genetic origin as the result of either being isolated from a particular geographic area, sharing particular chromosomes or alleles, or having been bred for multiple generations for a particular phenotype.

The term “conditions” when used in the context of invertebrate foraging behavior refers to environmental and biological factors that can influence invertebrate foraging behavior. Influences on invertebrate foraging behavior can cause, for example, increase, decrease or modification of a foraging behavior. Environmental factors encompass the physical environment such as temperature, pressure, light intensity, light position, and the like; components of the gaseous environment such as humidity, % oxygen, presence of a compound such as a drug or hormone, and the like; presence or absence of food; food quality; food quantity; food composition, and the structural makeup of the chamber in which the invertebrate is housed, including volume, particularly as it influences density of invertebrates, shape, composition of the chamber, and the like. Biological factors that can influence invertebrate foraging can include genetic factors including presence of particular alleles of genes or chromosomes, either naturally occurring or induced in the laboratory, biorhythmic factors such as time of day, relative activity level of an invertebrate, length of time an invertebrate has been active, and the like. Biological factors also include biochemical factors such as developmental and hormonal state of an invertebrate, fasting state of the invertebrate, presence in the invertebrate of an administered compound, gender and age.

As used herein, the term “polynucleotide molecule” refers to both deoxyribonucleic acid (DNA) and ribonucleic acid (RNA) molecules, and can optionally include one or more non-native nucleotides, having, for example, modifications to the base, the sugar, or the phosphate portion, or having a modified phosphodiester linkage. The term polynucleotide molecule includes both single-stranded and double-stranded polynucleotides, representing the sense strand, the anti-sense strand, or both, and includes linear, circular or branched molecules. A polynucleotide molecule of the invention can contain 2 or more nucleotides. Exemplary polynucleotide molecules include genomic DNA, cDNA, mRNA and oligonucleotides, corresponding to either the coding or non-coding portion of the molecule, and optionally containing sequences required for expression. A polynucleotide molecule of the invention, if desired, can additionally contain a detectable moiety, such as a radiolabel, a fluorochrome, a ferromagnetic substance, a luminescent tag or a binding agent such as biotin.

As used herein, the term “isolated,” when used in reference to a polynucleotide molecule, is intended to mean that the molecule is substantially removed or separated from components with which it is naturally associated, or otherwise modified by a human hand, thereby excluding polynucleotide molecules as they exist in nature. An isolated polynucleotide molecule of the invention can be in solution or suspension, or immobilized on a filter, glass slide, chip, culture plate or other solid support. The degree of purification of the polynucleotide molecule, and its physical form, can be determined by those skilled in the art depending on the intended use of the molecule.

As used herein, a “fragment” of a polynucleotide refers to a portion of a polynucleotide that includes at least 2 contiguous nucleotides of the polynucleotide. A mammalian polynucleotide can be substantially the same as an invertebrate polynucleotide, for example, when a fragment of a mammalian polynucleotide that encode a polypeptide domain corresponds to a polypeptide domain encoding fragment of an invertebrate polynucleotide. Such a fragment typically is encoded by at least 30 nucleotides, and the mammalian and invertebrate polynucleotides encoding that fragment share at least about 50% identity, at least about 60% identity, at least about 70% identity, at least about 80% identity, at least about 90% identity, at least about 95% identity or at least about 98% identity. Methods for determining that a fragment of a mammalian polynucleotide is substantially the same as an invertebrate polynucleotide or a fragment of an invertebrate polynucleotide include those described above for comparing mammalian and invertebrate polynucleotides. Such a fragment can be encoded by 30 or more nucleotides, for example, 45 or more nucleotides, 60 or more nucleotides, 90 or more nucleotides, 150 or more nucleotides, 210 or more nucleotides, or 300 or more nucleotides.

Biological functions retained by a fragment can include the ability to modulate ADHD or hypertension in a mammal, the ability to modulate invertebrate foraging, the ability to bind an antibody that binds to a full-length polypeptide which comprises the fragment, or an enzymatic or binding activity characteristic of the full length polypeptide.

The term “substantially the same” as used herein in reference to the relationship between a mammalian polynucleotide and an invertebrate polynucleotide refers to a mammalian polynucleotide or corresponding amino acid sequence that has a high degree of homology to an invertebrate polynucleotide or corresponding amino acid sequence and retains at least one function specific to the invertebrate polynucleotide or corresponding amino acid sequence. In the case of a nucleotide sequence, a first polynucleotide that is substantially the same as a second polynucleotide can selectively hybridize to a sequence complementary to the second polynucleotide under moderately stringent conditions or under highly stringent conditions. Therefore, a first polynucleotide molecule having substantially the same sequence compared to a second polynucleotide sequence can include, for example, one or more additions, deletions or substitutions with respect to the second sequence so long as it can selectively hybridize to a complement of that sequence.

In the case of an amino acid sequence, a first amino acid sequence that is substantially the same as a second amino acid sequence can contain minor modifications with respect to the second amino acid sequence, so long as the polypeptide containing the first amino acid sequence retains one or more functional activities exhibited by the whole polypeptide containing the second amino acid sequence.

Typically, a substantial similarity for a polypeptide or polynucleotide is represented by at least about 20% identity between mammalian and invertebrate sequences; mammalian and invertebrate sequences that are substantially the same can also share at least about 30% identity, at least about 40% identity, at least about 50% identity, at least about 60% identity, at least about 70% identity, at least about 80% identity, at least about 90% identity, at least about 95% identity, at least about 97% identity, or at least about 99% identity over the length of the two sequences being compared. Those skilled in the art know that two or more polypeptides having low overall sequence similarity can be substantially similar if the polypeptides have similar domains with substantial sequence similarity. For example, polypeptides having 20% overall identity can be substantially similar if the polypeptides contain one or more domains of substantial similarity. A larger number of similar domains between two or more polypeptides correlates with increased similarity. Therefore, substantial similarity can be identified according to sequence identity within similar domains of two or more polypeptides. Examples of methods for determining substantial similarity using sequence identity or a combination of sequence identity and similarity in domain structure are described below.

As used herein, the term “phenotype” refers to a set of detectable outward manifestations that are correlated with one or more allele or genetic loci of an organism. A set can include one, all, or a portion of all of the detectable outward manifestations. A phenotype can include detectable outward manifestations that are correlated with an entire genotype of an organism or a subset of alleles of a specific genotype. An example of a phenotype is Rover which is manifested, in part, by exploration of a broad area or long distance when an individual having the for^{R} allele perceives the presence of food or is in a fed state. The for^{S} allele is manifested in homozygous individuals, for example, as a phenotype which includes exploration of a limited area or short distance in the presence of food or when in a fed state.

As used herein, the term “antibody” is consistent with the definition of the term in the art and includes polyclonal and monoclonal antibodies, as well as antigen binding fragments of such antibodies. An antibody of the invention is characterized by having specific binding activity for a polypeptide associated with invertebrate foraging or with modulation of ADHD or hypertension in a mammal of at least about 1×10⁵ M⁻¹. Thus, Fab, F(ab′)₂, Fd and Fv fragments of a polypeptide-specific antibody of the invention, which retain specific binding activity, are included within the definition of an antibody. Methods of preparing polyclonal or monoclonal antibodies against polypeptides are well known in the art (see, for example, Harlow and Lane, Antibodies: A Laboratory Manual, Cold Spring Harbor Laboratory Press (1988)).

In addition, the term “antibody” as used herein includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be produced or obtained by methods known in the art, including constructing the antibodies using solid phase peptide synthesis, recombinant production, or screening combinatorial libraries consisting of variable heavy chains and variable light chains.

The invention provides a method of identifying a compound that modulates a disease associated with a nitric oxide/cGMP-dependent protein kinase network in a mammal. The method consists of administering a test compound to an invertebrate; and (b) measuring a foraging behavior of the invertebrate, wherein a compound that modulates the foraging behavior of the invertebrate is characterized as a compound that modulates a disease associated with a nitric oxide/cGMP-dependent protein kinase network in a mammal.

Thus, the invention provides, for example, a method of identifying a compound that modulates ADHD in a mammal. The method consists of (a)administering a test compound to an invertebrate; and (b) measuring a foraging behavior of the invertebrate, wherein a compound that modulates the foraging behavior of the invertebrate is characterized as a compound that modulates ADHD in a mammal.

The invention also provides a method of identifying a compound that modulates hypertension in a mammal. The method consists of (a) administering a test compound to an invertebrate; and (b) measuring a foraging behavior of the invertebrate, wherein a compound that modulates the foraging behavior of the invertebrate is characterized as a compound that modulates hypertension in a mammal.

A foraging behavior of an invertebrate can be observed as an action of the invertebrate in the presence of food or in a fed state. A behavior of an invertebrate that is preferentially or even exclusively displayed in the presence of food as compared to in the absence of food or in the fed state as compared to a starved state is desirable in the methods of the invention. Those behaviors for which a modulation can be quantified or reproducibly evaluated are preferred in the invention and include, for example, duration of search, frequency of searching, energy expended searching, distance of search, direction of search, rate of search, area of search, efficiency of search or diligence of search. One skilled in the art will be able to choose a particular behavior to be observed according to the features of the invertebrate as they affect behavior including, for example, means of locomotion such as flying or walking or developmental stage of the invertebrate.

An example of a foraging behavior useful in the methods of the invention is a phenotypic behavior associated with the foraging (for) gene. The for gene can be functionally characterized based on its influence on food search behavior in fruit flies as described for example in, Sokolowski, Behav. Genet. 10:291 (1980) and de Belle et al., Genetics 123:157 (1989). Individuals with a for^{R} allele are referred to as Rovers and explore a considerably wider area in the presence of food than do sitters who are homozygous for the for^{S} allele as described for example in de Belle et al., Heredity 59:73 (1987). Rovers and sitters display differences in foraging behavior in both the larval and adult life stages as described, for example, in Periera et al. Proc. Natl. Acad. Sci. USA 90:5044 (1993). Larval Rover and sitter strains do not significantly differ in activity in the absence of food but adults do differ somewhat. However, there is a food dependent differential effect on adult behavior in the presence of food.

An advantage of the invention is that the methods can be used to distinguish a compound that has a specific effect on ADHD, hypertension or other disease associated with a nitric oxide/cGMP-dependent protein kinase network in a mammal from a compound that has a non-specific effect. Because Rovers and sitters are both wild type forms that exist at appreciable frequencies in natural populations, the phenotypic behaviors displayed by both can be considered as normal, wild type behaviors. Changes in foraging behavior that cause a sitter to behave more like a Rover or a Rover to behave more like a sitter can be identified as having a specific effect on foraging. Therefore, the methods of the invention can be used to distinguish a compound that has a specific effect on foraging behavior from a compound that induces aberrant behavior by non-specific effects. In this regard, a compound that has a non-specific effect can be identified, for example, as a compound that in some way hinders or disables an invertebrate from foraging effectively. A compound identified by the methods of the invention as having a specific effect on foraging behavior, for example, by causing a sitter to behave more like a Rover or a Rover to behave more like a sitter, can be further identified as a compound having a specific effect on ADHD, hypertension or other disease associated with a nitric oxide/cGMP-dependent protein kinase network in a mammal.

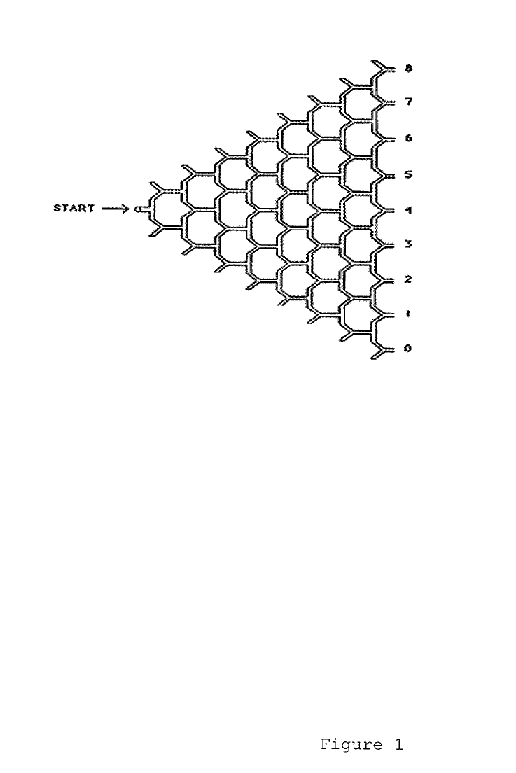

An example of a foraging behavior that can be evaluated in invertebrates is an area explored in the presence of food or in a fed state. Any method appropriate for the particular invertebrate can be used to introduce food. Foraging behavior of the invertebrate in response to food can be determined by a variety of means. For example, foraging behavior can be measured as a distance traversed in a fed state or a rate at which an identified distance is traversed. An exemplary method for determining foraging behavior in a population of adult Drosophila by determining a distance traveled in a defined amount of time is presented in Example I. As described in Example I, the number of flies that reach a collection tube in the chamber shown in FIG. 1 before a defined point in time can be summed and expressed as a percentage of the total number of flies tested to yield a foraging score. In addition, a foraging score for a population of flies can be determined by measuring the mean pathlength traversed by each fly in a defined amount of time. Foraging scores provide a convenient measure allowing quantitative determination of foraging behavior and comparison of foraging behaviors in different strains and/or different conditions.

Various manual and automated assays can be used to evaluate foraging behavior. For example, activity can be detected visually, either by direct observation or by photographic means. Additionally, an automated monitoring system can be used to detect motion at a specific distance from a food source including, for example, light beam detectors commonly employed in chromatographic fraction detectors or motion detectors commonly employed in security systems. As a further example, an infrared monitoring system, such as the infrared Drosophila Activity Monitoring System available from Trikinetics (described in M. Hamblen et al., J. Neurogen. 3:249 (1986)), can be used. Automated detection systems are advantageous when simultaneously evaluating activity in large numbers of invertebrates.

Those skilled in the art can determine an appropriate method to evaluate foraging behavior in a particular application of the method, depending on considerations such as the size and number of invertebrates, their normal activity level, the intended number of data points, and whether a quantitative or qualitative assessment of activity is desired.

An invertebrate of the invention can be an insect including for example a Drosophila melanogaster. Invertebrates are understood to refer to members of the division invertebrate. As disclosed herein, Drosophila melanogaster is an example of an invertebrate that exhibits foraging behavior that can be measured. Those skilled in the art understand that other Drosophila species are also likely to exhibit similar foraging behavior and express polynucleotides associated with foraging behavior, including D. simulans, D. virilis, D. pseudo obscura D. funebris, D. immigrans, D. repleta, D. affinis, D. saltans, D. sulphurigaster albostrigata and D. nasuta albomicans. Likewise, other flies, including, sand flies, mayflies, blowflies, flesh flies, face flies, houseflies, screw worm-flies, stable flies, mosquitos, northern cattle grub, and the like will also exhibit foraging behavior and express polynucleotides associated with foraging behavior.

Furthermore, insects other than flies can also exhibit foraging behavior and express polynucleotides associated with foraging behavior. For example, the invention can also be practiced with insects such as cockroaches, honeybees, wasps, termites, grasshoppers, moths, butterflies, fleas, lice, boll weevils, beetles, Apis mellifera, A. florea, A. cerana, Tenebrio molitor, Bombus terrestris, B. lapidarius, and members of Hydrocorisae.

Arthropods other than insects also can exhibit foraging behavior and express polynucleotides associated with foraging behavior. For example, the invention can also be practiced using arthropods such as scorpions, spiders, mites, crustaceans, centipedes and millipedes.

Due to the high degree of genetic similarity across invertebrate species, invertebrates other than arthropods can exhibit foraging behavior and express polynucleotides associated with foraging behavior. For example, foraging behaviors of nematodes such as those described for C. elegans in deBono and Bargmann, Cell 94:679-689 (1998) can be useful in the methods of the invention. Other invertebrates useful in the invention include, for example, flatworms, mollusks (e.g. Aplysia or Hermissenda), echinoderms and annelids all of which can exhibit foraging behavior and express polynucleotides associated with foraging behavior.

Those skilled in the art can determine, using the assays described herein, whether a particular invertebrate exhibits foraging behavior and expresses polynucleotides associated with foraging behavior and, therefore, would be applicable for use in the methods of the invention. The choice of invertebrate will also depend on additional factors, for example, the availability of the invertebrates, the normal activity levels of the invertebrates, the availability of molecular probes for polynucleotides associated with foraging behavior, the number of invertebrates and compounds one intends to use, the ease and cost of maintaining the invertebrates in a laboratory setting, the method of administering and type of compounds being tested, and the particular property being evaluated. Those skilled in the art can evaluate these factors in determining an appropriate invertebrate to use in the screening methods.

For example, if it is desired to evaluate gene expression in the methods of the invention, an invertebrate that is genetically well-characterized, such that homologs of genes associated with foraging behavior are known or can be readily determined, can be used. Thus, appropriate invertebrates in which to evaluate gene expression can include, for example, Drosophila and C. elegans. If it desired to evaluate behavioral properties in the methods of the invention, an invertebrate that exhibits one or more foraging behaviors, such as fruit flies, cockroaches, honeybees, wasps, moths, mosquitos, scorpions, and the like, can be used.

An invertebrate of the invention can be at any stage of development so long as a foraging behavior can be measured. For example, a Drosophila melanogaster can be an adult or larva. Larvae display the canonical foraging behavior as originally defined and used to identify the naturally occurring Rover and sitter alleles, show natural bimodal distribution in populations and map to the for locus [Sokolowski, M. Behav. Genet. 10: 291-302 (1980); de Belle et al., Genetics 123: 157-163 (1989)]. Adults have the advantage of being testable either as individuals or in a population assay, as well as having most of the genetically identifiable markers to permit construction and testing of various genotypes. In comparison to larvae, adults have much larger brains providing more convenient tissue samples for biochemical and molecular analysis (about 200,000 neurons vs. about 10,000) and can provide more easily enriched brain preparations due to ease of isolation of heads from adults compared to isolation of whole bodies from larvae. Differences between adult and larval flies are described, for example, in Ashburner, Drosophila: A Laboratory Handbook, Cold Spring Harbor Laboratory Press: Cold Spring Harbor, N.Y. (1989).

The methods of the invention can be practiced by contacting an invertebrate with a candidate compound and evaluating its effect on a foraging behavior. An appropriate method of administering a compound to an invertebrate can be determined by those skilled in the art and will depend, for example, on the type and developmental stage of the invertebrate, whether the invertebrate is active or inactive at the time of administering, whether the invertebrate is exhibiting a foraging behavior at the time of administering, the number of animals being assayed, and the chemical and biological properties of the compound (e.g. solubility, digestibility, bioavailability, stability and toxicity). For example, as shown in Example IV below, ritalin can be administered to Drosophila melanogaster by dissolving the drugs in fly food and providing the food to the flies.

A candidate compound can be administered to an invertebrate in a single dose, or in multiple doses. It is expected that the modulation of invertebrate foraging behavior will be dose dependent. An effective amount of a compound used in the methods of the invention can be determined by those skilled in the art, and can depend on the chemical and biological properties of the compound and the method of contacting the invertebrate. Exemplary concentration ranges to test include from about 10 μg/ml to about 500 mg/ml, such as from about 100 μg/ml to 250 mg/ml, including from about 1 mg/ml to 200 mg/ml.

The appropriate time and duration to administer a compound can be determined by those skilled in the art depending on the application of the method. For example, it may be desirable to administer a compound prior to introducing food to the invertebrate, in the presence of food for a defined duration, or continuously in the presence of food, depending on the foraging behavior being evaluated, the mode of administration, the rate at which the drug compound has an effect, the duration of the compound's affect and the desired effect of the compound. If desired, a candidate compound can be combined with, or dissolved in, an agent that facilitates uptake of the compound by the invertebrate, such as an organic solvent, for example, DMSO or ethanol; or an aqueous solvent, for example, water or a buffered aqueous solution; or food. One skilled in the art will know the appropriate formulation based on the method of delivery to be used.

A compound used to contact the invertebrate can be identified as any molecule that potentially alters foraging. Additionally, a compound to be used in the methods of the invention can be identified based on presumed or predicted activity in ADHD, hypertension, or other disease associated with a NO/cGMP-dependent kinase network in a mammal as indicated for example by molecular properties, interactions observed at the molecular or cellular level, clinical evidence, or other empirical evidence known to one skilled in the art to be predictive of such activities.

In regard to molecular interactions, a compound for use in the methods of the invention can be identified based on its ability to alter polynucleotide or polypeptide expression or activity. For example, a compound can directly interact with a gene promoter; can interact with transcription factors that regulate polynucleotide expression; can bind to or cleave a polynucleotide transcript (e.g. antisense polynucleotides or ribozymes); can alter half-life of the transcript; or can itself be an expressible polynucleotide associated with invertebrate foraging or with ADHD, hypertension or a disease associated with a nitric oxide/cGMP-dependent protein kinase network in a mammal. A compound can also be identified or designed based on inhibition or activation of a cellular component known to be involved in ADHD, hypertension or a disease associated with a nitric oxide/cGMP-dependent protein kinase network in a mammal. For example, a compound can specifically bind to a polypeptide and alter its activity or half-life or a compound can bind to a substrate or modulator of a polypeptide.

A candidate compound can be a naturally occurring macromolecule, such as a peptide, polynucleotide, carbohydrate, lipid, or any combination thereof, or a partially or completely synthetic derivative, analog or mimetic of such a macromolecule. A candidate compound can also be a small organic or inorganic molecule, either naturally occurring, or prepared partly or completely by synthetic methods.

The methods of the invention can be performed with a single compound or by screening a number of compounds, including for example, a library of compounds. The number of different compounds to screen using the methods of the invention can be determined by those skilled in the art depending on the application of the method. For example, a smaller number of candidate compounds would generally be used if the type of compound that is likely to modulate foraging behavior in an invertebrate, or ADHD, hypertension or other disease associated with nitric oxide/cGMP-dependent protein kinase network in a mammal, is known or can be predicted, such as when derivatives of a lead compound are being tested. However, when the type of compound that is likely to modulate foraging behavior is unknown, it is generally understood that the larger the number of candidate compounds screened, the greater the likelihood of identifying a compound that modulates foraging behavior. Therefore, the methods of the invention can employ screening individual compounds separately or populations of compounds including small populations and large or diverse populations, to identify a compound that modulates foraging behavior, and thereby also modulates ADHD, hypertension or other disease associated with a nitric oxide/cGMP-dependent protein kinase network in a mammal.

Methods for producing libraries of compounds to use in the methods of the invention, including chemical or biological molecules such as simple or complex organic molecules, metal-containing compounds, carbohydrates, peptides, polypeptides, peptidomimetics, carbohydrates, lipids, polynucleotides, antibodies and the like, are well known in the art (see, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czarnik, eds., Combinatorial Chemistry: Synthesis and Application, John Wiley & Sons, New York (1997), Gold et al., U.S. Pat. Nos. 5,475,096 (1995), 5,789,157 (1998), and 5,270,163 (1993)). The advantage of using such a combinatorial library is that molecules do not have to be individually generated to identify a compound that modulates foraging behavior in an invertebrate. Also, no prior knowledge of the exact characteristics of molecular components associated with foraging behavior in an invertebrate or ADHD, hypertension, or a disease associated with a NO/cGMP-dependent kinase network in a mammal is required when using a combinatorial library. Libraries containing large numbers of natural and synthetic compounds also can be individually synthesized or obtained from commercial sources.

Following contacting an invertebrate with a compound, a foraging behavior can be evaluated and a determination made as to the effect of the compound on the foraging behavior. A compound can have a variety of effects on foraging including, for example, changing a foraging behavior, increasing a foraging behavior, or. decreasing a foraging behavior. A changed foraging behavior that occurs following administration of a compound can be observed, for example, as a new strategy of foraging not observed in the absence of the compound. An increased foraging behavior can be observed, for example, as increased duration of search, frequency of searching, energy expended searching, distance of search, rate of search, area of search or diligence of search.

A compound can also have the effect of changing a foraging phenotype. In this regard, a foraging phenotype can be changed such that the phenotype is decreased. In this regard, a compound identified by the methods of the invention can decrease a foraging phenotype in a sitter. For example, a sitter which normally forages a relatively short distance for food can be changed to forage a greater distance by a compound administered in the methods of the invention. A compound identified by the methods of the invention can also decrease a foraging phenotype of a Rover. For example, administration of a compound to a Rover who normally explores a large distance in the presence of food, can induce the Rover to decrease the distance traveled in the presence of food. A compound can change a foraging phenotype by increasing a foraging phenotype. For example, a compound can further decrease foraging behavior in a sitter or further increase foraging behavior in a Rover.

A compound of the invention can modulate the expression or activity of one or more mammalian polypeptides. A compound that modulates the expression of a polypeptide can, for example, increase or decrease the quantity of the polypeptide produced from a gene or other polynucleotide. For example, a compound can affect the transcription of a DNA or the translation of an RNA encoding the polypeptide. An activity of a polypeptide that when modulated can cause modulation of foraging behavior in an invertebrate can include stability to proteolysis or other form of cellular inactivation, binding activity with a ligand, enzymatic activity, binding activity with other cellular components, or susceptibility to post-translational modifications such as phosphorylation, prenylation, iso prenylation and the like.

As described herein, a compound that modulates invertebrate foraging behavior can also modulate ADHD, hypertension or other disease associated with a nitric oxide/cGMP-dependent protein kinase network in a mammal. A compound identified by the methods of the invention as modulating foraging behavior in an invertebrate can decrease the severity of ADHD, hypertension or other disease associated with a nitric oxide/cGMP-dependent protein kinase network in a mammal or a symptom thereof. For example, a compound that decreases foraging behavior of an invertebrate can decrease the severity of ADHD, hypertension or other disease associated with a nitric oxide/cGMP-dependent protein kinase network in a mammal or a symptom thereof. Thus, a compound that decreases foraging behavior of a Rover can decrease the severity of ADHD, hypertension or other disease associated with a nitric oxide/cGMP-dependent protein kinase network in a mammal or a symptom thereof. In addition, a compound that increases foraging behavior of an invertebrate can decrease the severity of ADHD, hypertension or other disease associated with a nitric oxide/cGMP-dependent protein kinase network in a mammal or a symptom thereof. Thus, a compound that increases foraging behavior of a sitter can decrease the severity of ADHD, hypertension or other disease associated with a nitric oxide/cGMP-dependent protein kinase network in a mammal or a symptom thereof.

A compound of the invention can modulate ADHD or hypertension in any mammal including, for example, a human. Modulation of ADHD can be identified as any change in severity of ADHD or change in a symptom of ADHD including, for example, a decrease or removal thereof. For example, a compound identified by the methods of the invention to modulate foraging behavior in an invertebrate can also be identified as modulating distract ability, impulsivity, or hyperactivity in a human having ADHD. Modulation of hypertension can similarly be identified as any change in severity of hypertension or change in a symptom of hypertension including, for example, a decrease or removal thereof. Thus, a compound identified by the methods of the invention to modulate foraging behavior in an invertebrate can also be identified as modulating abnormally high blood pressure; drowsiness; confusion; headache; nausea; loss of vision; or increased risk for arteriosclerosis, angina pectoris, sudden death, stroke, dissecting aortic aneurysm, intra cerebral hemorrhage, rupture of the myocardial wall or artherothrombotic occlusion of the abdominal aorta in an individual having hypertension.

A compound identified by the methods of the invention can modulate other diseases associated with a NO/cGMP-dependent protein kinase network in a mammal including, for example, diabetes (Tooke, J. Diabetes Complications 14:197-200 (2000)); atherosclerosis (Bundy, et al., Gen. Pharmacol. 34:73-84 (2000)); coronary artery disease (Ijem and Granlie, S. D. J. Med. 53:489-491 (2000)); cirrhosis (Knotek et al., Can. J. Gastroenterol. 14:112D-121D (2000)).; asthma and bronchitis Ratjen, Pediatr. Allergy Immunol. 11:230-235 (2000)); uveitis, retinopathy, macular degeneration, glaucoma and myopia (Chiou et al., J. Ocul. Pharmacol. Ther. 16:407-418 (2000)); nonalcoholic steatohepatitis associated with obesity (Garcia-Monzon et al., J. Hepatol 33:716-724 (2000)); Duchenne muscular dystrophy (Sander et al., Proc. Natl. Acad. Sci. USA 97:13818-13823 (2000)); and sleep apnea (Kato, Circulation 102:2607-2610 (2000)).

A method of identifying a compound that modulates a disease associated with nitric oxide/cGMP-dependent protein kinase network in a mammal can consist of (a) administering a test compound to an invertebrate; (b) measuring an expression level for one or more polynucleotides in the invertebrate; and (c) comparing the expression level for one or more polynucleotides in the invertebrate to an expression level of one or more polynucleotides in a reference invertebrate, a compound having the effect of modulating the expression level of one or more polynucleotides associated with invertebrate foraging behavior in the test invertebrate relative to the reference invertebrate is identified as a compound that modulates a disease associated with nitric oxide/cGMP-dependent protein kinase network in a mammal.

Therefore, a method of identifying a compound that modulates ADHD in a mammal can consist of (a) administering a test compound to an invertebrate; (b) measuring an expression level for one or more polynucleotides in the invertebrate; and (c) comparing the expression level for one or more polynucleotides in the invertebrate to an expression level of one or more polynucleotides in a reference invertebrate, a compound having the effect of modulating the expression level of one or more polynucleotides associated with invertebrate foraging behavior in the test invertebrate relative to the reference invertebrate is identified as a compound that modulates ADHD in a mammal.

Additionally, a method of identifying a compound that modulates hypertension in a mammal can consist of (a) administering a test compound to an invertebrate; (b) measuring an expression level of one or more polynucleotides in the invertebrate; and (c) comparing the expression level of one or more polynucleotides in the invertebrate to an expression level of one or more polynucleotides in a reference invertebrate, a compound having the effect of modulating the expression level of one or more polynucleotides associated with invertebrate foraging behavior in the test invertebrate relative to the reference invertebrate is identified as a compound that modulates hypertension in a mammal.

One or more polynucleotides having modulated expression levels in the methods of the invention can be selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104. A mammalian polynucleotide can be identified having substantially the same sequence as a polynucleotide sequence selected from the above group.

According to the invention a compound that has the effect of increasing expression of one or more polynucleotides in an invertebrate relative to a reference invertebrate can be identified as a compound that decreases severity of ADHD or hypertension. Additionally, a compound that has the effect of decreasing expression of a specific polynucleotide in a test invertebrate relative to a reference invertebrate can be identified as a compound that decreases a symptom of ADHD or hypertension.

A variety of assays well known in the art can be used to evaluate expression of particular polynucleotides or polypeptides associated with invertebrate foraging behavior including, for example, the invertebrate polynucleotides or polypeptides comprising NOS: 1-105. Assays that detect mRNA expression generally involve hybridization of a detectable agent, such as a complementary primer or probe, to the polynucleotide molecule. Such assays include, for example, RNA or dot blot analysis, primer extension, RNase protection assays, reverse-transcription PCR, competitive PCR, real-time quantitative PCR (TaqMan PCR), polynucleotide array analysis, and the like.

Additionally, constructs containing the promoter of a gene associated with foraging behavior in an invertebrate or with ADHD, hypertension, or other disease associated with a nitric oxide/cGMP-dependent protein kinase network in a mammal can be functionally fused to a reporter gene (e.g. β-galactosidase, green fluorescent protein, luciferase) using known methods, and used to generate transgenic invertebrates. Such transgenic invertebrates can be used in the methods of the invention wherein expression of the reporter gene can be a marker for expression of a polynucleotide that modulates ADHD, hypertension or other disease associated with a nitric oxide/cGMP-dependent protein kinase network in a mammal.

Those skilled in the art will appreciate that the methods of the invention can be practiced in the absence of knowledge of the sequence or function of the polynucleotides associated with ADHD, hypertension, or other disease associated with a nitric oxide/cGMP-dependent protein kinase network in a mammal or a foraging behavior in an invertebrate. Expression of such polynucleotides can thus be evaluated using assays that examine overall patterns of polynucleotide expression characteristic of a foraging behavior. It will be understood that as these polynucleotides are identified or sequenced, specific probes, primers, antibodies and other binding agents can made by methods well known in the art and used to evaluate their expression more specifically using any of the above detection methods.

One assay to examine patterns of expression of polynucleotides associated with a foraging behavior or polynucleotides that modulate the mammalian vestibular system, that does not require prior knowledge of their sequence, is mRNA differential display, which is described, for example, in Cirelli et al., Mol. Brain Res. 56:293 (1998) and Liang and Pardee, Mol. Biotech. 10:261-7 (1998). In such a method, RNA from the animal is reverse-transcribed and amplified by PCR using a particular combination of arbitrary primers. A detectable label, such as an enzyme, biotin, fluorescent dye or a radiolabel, is incorporated into the amplification products. The labeled products are then separated by size, such as on acrylamide gels, and detected by any method appropriate for detecting the label, including autoradiography, phosphoimaging or the like.

Such a method allows concurrent examination of expression of thousands of RNA species. Methods for determining which RNA species correspond to a polynucleotide associated with a foraging behavior, are disclosed herein, for example, comparing polynucleotide expression levels in invertebrates that exhibit different foraging behavior. It can be readily determined whether a particular compound alters a pattern of polynucleotide expression, such as by increasing or decreasing the intensity of bands corresponding to polynucleotides associated with a foraging behavior.

A further assay to examine patterns of expression of polynucleotides is array analysis, in which polynucleotides representative of all or a portion of the genome of an invertebrate or mammal, or representative of all or a portion of expressed polynucleotides of an invertebrate or mammal, are attached to a solid support, such as a filter, glass slide, chip or culture plate. Detectably labeled probes, such as cDNA probes, are then prepared from mRNA of an animal, and hybridized to the array to generate a characteristic, reproducible pattern of expression associated with, for example, a foraging behavior. It can be readily determined whether a particular candidate compound alters this pattern of polynucleotide expression, by detecting an increase or decrease in the amount of probe hybridized at one or more location on the array.

An expression profile used in the methods of the invention can be any read-out that provides a qualitative or quantitative indication of the expression or activity of a single polynucleotide or polypeptide, or of multiple polynucleotides or polypeptides. An expression profile can, for example, indicate the expression or activity of one, or of least 2, at least 5, at least 10, at least 20, at least 50, at least 100, at least 265, or more polynucleotides or polypeptides. An expression profile can, for example, indicate the expression or activity in a mammal of mammalian homologs of one or more polynucleotides or polypeptides associated with invertebrate foraging behavior. An expression profile can also, for example, indicate the expression or activity in an invertebrate of one more polynucleotides or polypeptides associated with invertebrate foraging behavior. An expression profile can indicate modulated expression or activity of one, a few, many, or all of the polypeptides or polynucleotides in the profile. Therefore, the methods of the invention can be used to identify modulated expression of 1 or more polynucleotide or polypeptide including, for example, 2 or more, 3 or more, 4 or more, 5 or more, 8 or more, 10 or more, 12 or more, 15 or more, 20 or more, 25 or more, or 50 or more polynucleotides or polypeptides.

The methods of the invention can be used to identify expression levels of any subset of polynucleotides or polypeptides desired to characterize a disease associated with NO/cGMP-dependent protein kinase network. Such a subset of polynucleotides or polypeptides can be identified by the methods of the invention. In addition a subset of polynucleotides or polypeptides, that has been previously identified, can be isolated and used in the methods of the invention or the methods can be directed to detect only members of the subset within a larger population. A subset of polynucleotides or polypeptides can be chosen based on functional linkage of the polynucleotides or polypeptides including, for example, interaction in a signal transduction system or a metabolic system; physical linkage of the polynucleotides including, for example, proximity on a chromosome; or correlated co-expression.

An expression profile can also be a quantitative or qualitative measure of expression of polypeptides encoded by one or more polynucleotides. Such assays generally involve binding of a detectable agent, such as an antibody or selective binding agent, to the polypeptide in a sample of cells or tissue from the animal. Protein assays include, for example, immunohistochemistry, immunofluorescence, ELISA assays, immunoprecipitation, immunoblot or other protein-blot analysis, and the like. Additional methods include two-dimensional gel electrophoresis, MALDI-TOF mass spectrometry, and ProteinChipm/SELDI mass spectrometry technology.

An expression profile can also be a direct or indirect measure of the biological activity of polypeptides encoded by one or more polynucleotides. A direct measure of the biological activity of a polypeptide can be, for example, a measure of its enzymatic activity, using an assay indicative of such enzymatic activity. An indirect measure of the biological activity of a polypeptide can be its state of modification (e.g. phosphorylation, glycosylation, or proteolytic modification) or localization (e.g. nuclear or cytoplasmic), where the particular modification or localization is indicative of biological activity. A further indirect measure of the biological activity of a polypeptide can be the abundance of a substrate or metabolite of the polypeptide, such as a neurotransmitter, where the abundance of the substrate or metabolite is indicative of the biological activity of the polypeptide. Appropriate assays for measuring enzyme activity, polypeptide modifications, or amounts of substrates or product of an enzymatic polypeptide, can be determined by one skilled in the art based on the biological activity of the particular polypeptide.

An appropriate method to use in determining an expression profile can be determined by those skilled in the art, and will depend, for example, on the number of polynucleotides being profiled; whether the method is performed in vivo or in a sample; the type of sample obtained; whether the assay is performed manually or is automated; the biological activity of the encoded polypeptide; the abundance of the transcript, protein, substrate or metabolite being detected; and the desired sensitivity, reproducibility and speed of the method.

An expression profile can be established in vivo, such as by diagnostic imaging procedures using detectably labeled antibodies or other binding molecules, or from a sample obtained from an individual. As changes in polynucleotide expression in the brain are likely to be most relevant to modulation of foraging behavior, appropriate samples can contain neural tissue, cells derived from neural tissues, or extracellular medium surrounding neural tissues, in which polypeptides to be detected or their metabolites are present. Thus, an appropriate sample for establishing an expression profile in humans can be, for example, cerebrospinal fluid, whereas in laboratory animals an appropriate sample can be, for example, a biopsy of the brain.

However, expression of polynucleotides can also be modulated in tissues other than neural tissue, and polypeptides or their metabolites can be secreted into bodily fluids. In particular, in the case of genetic disorders, an alteration in gene expression or function can be manifest in other cells in the body. Alternatively, a genetic disorder can be determined using any cell that contains genomic DNA, by detecting a mutation such as an insertion, deletion or modification of a gene associated with invertebrate foraging or a gene that modulates a mammalian vestibular system. An expression profile or presence of a genetic mutation can be determined from any convenient cell or fluid sample from the body, including blood, lymph, urine, breast milk, skin, hair follicles, cervix or cheek. Additionally, cells can readily be obtained using slightly more invasive procedures, such as punch biopsies of the breast or muscle, from the bone marrow or, during surgery, from essentially any organ or tissue of the body.

An expression profile can also be determined from cells in culture. These cells can be immortalized cells from a selected individual invertebrate or mammal, or can be cells from any known established invertebrate or mammalian cell line, such as those available from ATCC (Mannassas, Va.). The expression profile of these cells can be measured, for example, in the absence and presence of a compound. A compound that modulates the expression of an invertebrate polynucleotide associated with foraging behavior or of a mammalian polynucleotide substantially the same as an invertebrate polynucleotide associated with foraging behavior can be a compound that modulates ADHD, hypertension, or other disease associated with a NO/cGMP-dependent protein kinase network.

Following identification of patterns of polynucleotide or polypeptide expression, those skilled in the art can determine the sequence and if desired clone the polynucleotide using standard molecular biology approaches. For example, a polynucleotide identified by differential display can be isolated and sequenced, or used to probe a library to identify the corresponding cDNA or genomic DNA. Likewise, a polynucleotide from an array can be identified based on its known position on the array and used to amplify or clone the corresponding cDNA or genomic DNA.

If desired, any of the expression and activity assays described above can be used in combination, either sequentially or simultaneously. Such assays can also be partially or completely automated, using methods known in the art.

Samples of the invertebrate collected for measuring polynucleotide expression levels can include any organ known or suspected of influencing foraging. Exemplary organs can be found in the head, neck, legs and antennae, and include, for example, the brain or nervous system. Samples can be collected from an invertebrate at various occasions, including before and/or after feeding, before and/or after administration of a compound, or before and/or after participating in a measurement of a foraging behavior. Typically, samples are collected under the same conditions as the conditions that foraging measurements are carried out, for example at about the same time of day, about the same amount of time after feeding, about the same environmental conditions, and the like. Samples can also be collected immediately following measurement of foraging behavior. For example, samples from a first and a second invertebrate can be collected immediately after subjecting the first and second invertebrates to conditions in which the first invertebrate exhibits a foraging behavior different than a foraging behavior exhibited by the second invertebrate. Typically, a time period considered immediately after an exhibited behavior is less than 5 minutes after measuring foraging behavior, but the time period can be any amount of time considered by one skilled in the art to be immediate relative to the period of time in which expression of a polynucleotide or polypeptide of interest can change. In this regard, a polynucleotide or polypeptide whose expression changes rapidly will require shorter times between observation of a foraging behavior and measurement of polynucleotide expression compared to a polynucleotide or polypeptide having a slower rate of change in expression level.

Evaluation of expression can involve sacrificing the animal at a selected time and homogenizing the entire animal, or a portion thereof, such as the brain or a neuronal tissue. One or more polynucleotide or polypeptide molecules can then be extracted therefrom. Alternatively, such assays can be performed with a polypeptide or polynucleotide extracted from a biopsied tissue of an invertebrate.

According to the methods of the invention polynucleotide or polypeptide expression levels in an invertebrate can be compared to polynucleotide or polypeptide expression levels in a reference invertebrate. A reference invertebrate used in the methods of the invention can be chosen based on a variety of factors that can influence foraging behavior including, for example, strain, genotype, age, gender, developmental stage, presence or absence of defined mutations or polymorphisms, exposure to a compound or lack of exposure to a compound, or having been subjected to a particular condition or set of conditions during a foraging assay. For example, an invertebrate exposed to a compound can be compared to a reference invertebrate that has not been exposed to the compound. Alternatively, a reference invertebrate can be exposed to the compound to which the test invertebrate was exposed.

It is possible that an invertebrate used in the methods of the invention can exhibit substantially the same foraging behavior as a reference invertebrate before a compound is administered. It is also possible that an invertebrate used in the methods of the invention can exhibit a different foraging behavior from a reference invertebrate before a compound is administered. Following administration of a compound, an invertebrate may display a foraging behavior that is substantially the same or different from a foraging behavior displayed by a reference invertebrate that has been either exposed to the same compound, to another compound or not exposed to the compound. Additionally, an invertebrate can be its own reference and polynucleotide or polypeptide expression can be measured at different times or under different conditions.

One skilled in the art can choose a reference invertebrate for use in the methods of the invention according to factors suspected or known to affect the desired comparison. In cases where differences in conditions or effect of a compound are to be determined, it can be advantageous to use a reference invertebrate that is similar to the invertebrate being tested. In this regard, differential expression can be determined for invertebrates of the same strain that have been subjected to conditions in which a first group of members of a strain exhibit a foraging behavior different from the foraging behavior of a second group of members of the strain. Such conditions can be, for example, administration of a compound, presence or absence of food, feeding or starvation, and the like. Differential expression is then determined by measuring expression levels in the two groups and identifying polynucleotides expressed at significantly different levels.

Polynucleotides or polypeptides that are expressed at significantly different levels can be termed differentially expressed. Significantly different levels are levels that vary from each other by a diagnostic amount. A diagnostic amount can be, for example, an amount that is greater than the variability of expression levels between invertebrates that ideally would have identical expression levels (i.e., having identical genetic makeup, age, gender, raised under identical conditions, and the like). In such a situation, a significantly different level can be a difference that is greater than the mean difference observed between expression levels, or greater than the largest expression level difference observed between most or all polynucleotides in the ideally identical organisms. Alternatively, significantly different levels can be based on the composite variability of polynucleotide expression levels between two or more strains. For example, the mean or median difference between polynucleotide expression levels can be determined between a large number of different strains. Any difference in expression that is greater than the mean or median difference can be considered differentially expressed. Significantly different levels of expression can be identified as a minimum percent change including, for example, at least a 100% change, at least a 75% change, at least a 50% change, at least a 25% change or at least a 10% change in expression level or lower. Significantly different levels of expression that are even larger can be observed in the methods of the invention and can be identified as a minimum fold change, including for example, at least 2 fold change, at least a 3 fold change, at least a 4 fold change, at least a 5 fold change, at least a 10 fold change in expression level or higher. Other reference levels defining a significant difference can be determined by one of skill in the art according to the desired comparison between two or more invertebrates.

A gene that is differentially expressed in two invertebrate groups that exhibit different foraging behaviors can be considered a gene associated with invertebrate foraging behavior. It is understood that a polynucleotide associated with invertebrate foraging behavior is a polynucleotide whose expression is correlated with modulation of invertebrate foraging behavior. For example, a polynucleotide associated with foraging behavior can be a polynucleotide identified as more highly expressed in a Rover than in a sitter. The sequence and function of such an associated polynucleotide can be previously known or unknown. Exemplary invertebrate polynucleotides associated with invertebrate foraging behavior are foraging/dg2 (SEQ ID NO: 47); alcohol dehydrogenase (SEQ ID NO: 75); inositol polyphosphatel 1-phosphatase (SEQ ID NO: 48); inositol 1,4,5-tris-phosphate receptor (SEQ ID NO: 49); Dead Box-1 (SEQ ID NO: 50); CNS-specific protein Noe (SEQ ID NO: 51); cellular repressor of E1A-stimulated genes (SEQ ID NO: 77); 14-3-3 ε (SEQ ID NO: 52); casein kinase II α subunit (SEQ ID NO: 53); mRNA sequence similar to syntaxin I (SEQ ID NO: 54); ADP/ATP translocase/sesB (SEQ ID NO: 55); mitochondrial porin (SEQ ID NO: 56); neuron specific zinc finger transcription factor (scratch) (SEQ ID NO: 57); ecdysone-regulated (E93)(SEQ ID NO: 58); centrosomal and chromosomal factor (ccf) (SEQ ID NO: 59); activin β precursor (SEQ ID NO: 79); dynamin-like (SEQ ID NO: 81); paramyosin; mitochondrial ATP synthase α subunit; Fas-associated factor (FFAF)(SEQ ID NO: 87); lamin precursor (SEQ ID NO: 89); 18S, 5.8S, 2S and 28S rRNA genes (SEQ ID NO: 60); and ribosomal protein S6 gene (SEQ ID NO: 61). Additional exemplary polynucleotides associated with invertebrate foraging behavior are polynucleotides that contain a polynucleotide sequence selected from SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 or SEQ ID NO: 104. Such a polynucleotide associated with invertebrate foraging behavior can be substantially the same as at least one mammalian polynucleotide that modulates ADHD, hypertension or other disease associated with a NO/cGMP-dependent protein kinase network. Therefore, polynucleotides selected from SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO:95, SEQ ID NO: 100, SEQ ID NO: 102 and SEQ ID NO: 104. can be substantially the same as polynucleotides that modulate ADHD, hypertension or other disease associated with NO/cGMP-dependent protein kinase network.

The invention provides a method of identifying a polynucleotide that correlates with a disease associated with nitric oxide/cGMP-dependent protein kinase network in a mammal. The method consists of (a) obtaining a first and a second strain of an invertebrate; (b) subjecting the first and second invertebrate strains to conditions in which the first strain exhibits a foraging behavior different than a foraging behavior exhibited by the second strain; (c) measuring an expression level for one or more polynucleotides in the first and second strains, and (d) identifying one or more polynucleotides that are differentially expressed in the first strain relative to the second strain, wherein a mammalian polynucleotide comprising substantially the same nucleic acid sequence as the one or more differentially expressed polynucleotides correlates with ADHD in a mammal.

Therefore, the invention provides a method of identifying a polynucleotide that correlates with ADHD in a mammal. The method consists of (a) obtaining a first and a second strain of an invertebrate; (b) subjecting the first and second invertebrate strains to conditions in which the first strain exhibits a foraging behavior different than a foraging behavior exhibited by the second strain; (c) measuring an expression level for one or more polynucleotides in the first and second strains, and (d) identifying one or more polynucleotides that are differentially expressed in the first strain relative to the second strain, wherein a mammalian polynucleotide comprising substantially the same nucleic acid sequence as the one or more differentially expressed polynucleotides correlates with ADHD in a mammal.

The invention also provides a method of identifying a polynucleotide that correlates with hypertension. The method consists of (a) obtaining a first and a second strain of an invertebrate; (b) subjecting the first and second invertebrate strains to conditions in which the first strain exhibits a foraging behavior different than a foraging behavior exhibited by the second strain; (c) measuring an expression level for one or more polynucleotides in the first and second strains, and (d) identifying one or more polynucleotides that are differentially expressed in the first strain relative to the second strain, wherein a mammalian polynucleotide comprising substantially the same nucleic acid sequence as the one or more differentially expressed polynucleotides correlates with hypertension in a mammal.

Conditions in which a first invertebrate strain can exhibit a foraging behavior different than a foraging behavior in a second invertebrate strain include, for example, environmental or biorhythmic factors that, when imposed on two different invertebrate strains, result in the two strains exhibiting dissimilar foraging behavior. Environmental factors include, for example, the physical environment such as temperature, pressure, light intensity, light quality, time of day, presence of food and the like; components of the gaseous environment such as humidity, % oxygen, presence of a compound such as a drug or hormone, and the like; and the structural makeup of the chamber in which the invertebrate is housed, including volume, particularly as it influences density of invertebrates, shape, composition of the chamber, and the like. Biological factors that can influence invertebrate foraging can include, for example, genetic factors such as presence of a particular allele, mutations that are either naturally occurring or induced in the laboratory; biorhythmic factors such as time of day, relative activity level of an invertebrate, length of time an invertebrate has been active, and the like; and biochemical factors such as developmental and hormonal state of an invertebrate, fasting state of the invertebrate, presence in the invertebrate of an administered compound, and the like. Further included are factors such as gender and age of the invertebrate.

Typically, foraging behavior experiments are carried out on adult invertebrates during the daytime, at least about two hours after sunrise and at least about two hours before sunset, and at least two hours after invertebrates have been at a relatively increased level of activity. Typical environmental conditions are about 22° C., 1 atmosphere, at ambient humidity and in a darkened room. An exemplary chamber is described in Example I and shown in FIG. 1.

Determination of a foraging behavior of a first invertebrate that is different when compared to a foraging behavior of a second invertebrate can be accomplished by analysis of foraging behavior for the two invertebrates. For example, in order for a foraging behavior of a first invertebrate to be different from a foraging behavior of a second invertebrate, the mean foraging measurement, typically termed the foraging score, of the first invertebrate will differ from the foraging score of the second invertebrate strain if a pairwise t-test of two scores is significantly different at the 0.05 level, or if multiple pairwise comparisons between strains are significantly different after applying a correction for experiment wise-error. A significantly different score refers to a score that is different by a statistically meaningful amount. In cases where foraging scores for populations of invertebrates are compared, two foraging scores are considered different if a first mean foraging score is not within a desired region of the probability distribution of the second foraging score. For example, a first mean foraging score can be different if it is not within the 80% probable region of a probability distribution of the second foraging score, or within the 85%, 90%, 95% or 98% probable region of the distribution of the second foraging score. Correspondingly, foraging scores considered to be substantially the same are foraging scores that do not differ by a more than a desired standard deviation or are within a desired probable region of a probability distribution. Methods for the determination of mean, standard deviation and characteristics of normal distributions are known in the art as demonstrated by texts such as Biostatistical Analysis 4th ed., Zar, Prentice-Hall Inc. (1999).

A mammalian polynucleotide or polypeptide that is associated with ADHD, hypertension or a disease associated with a NO/cGMP-dependent protein kinase network in a mammal can be identified by sequence homology with a polypeptide or polynucleotide of an invertebrate that is modulated in association with altered foraging behavior. A mammalian sequence that is substantially the same as an invertebrate sequence can be identified as a mammalian nucleic acid or corresponding amino acid sequence that has a high degree of homology to an invertebrate nucleic acid or corresponding amino acid sequence and at least one similar function.

According to the methods of the invention increased expression of a mammalian polynucleotide can correlate with an increase or decrease in severity of ADHD or hypertension. In addition decreased expression of a mammalian polynucleotide can correlate with an increase or decrease in severity of ADHD or hypertension. Thus, according to the methods of the invention, one or more polynucleotides that are differentially expressed in a first invertebrate strain relative to a second invertebrate strain can have increased expression and a mammalian polynucleotide having substantially the same sequence can have increased expression when involved in ADHD or hypertension in a mammal. Alternatively, a mammalian polynucleotide having substantially the same sequence as an invertebrate polynucleotide that demonstrates increased expression can have decreased expression when involved in ADHD or hypertension in a mammal. Additionally, one or more polynucleotides that are differentially expressed in a first invertebrate strain relative to a second invertebrate strain can have decreased expression and a mammalian polynucleotide having substantially the same sequence can have decreased expression when involved in ADHD or hypertension in a mammal. Alternatively, a mammalian polynucleotide having substantially the same sequence as an invertebrate polynucleotide that demonstrates decreased expression can have increased expression when involved in ADHD or hypertension in a mammal.

Polynucleotides that have substantially the same sequence can be identified, for example, by hybridization techniques where the polynucleotide molecules selectively hybridize via complementary base pairing under moderately stringent conditions or under highly stringent conditions. Stringency depends on a variety of factors including, for example, temperature, concentration of probe and/or target polynucleotide, ionic strength and pH. As known to those of skill in the art, the stability of hybrids is reflected in the melting temperature (Tₘ) of the hybrids. Typically, the hybridization reaction is performed under conditions of lower stringency, followed by washes of varying, but higher, stringency.

Moderately stringent hybridization refers to conditions that permit a target-polynucleotide to bind a complementary polynucleotide that has about 60% identity, preferably about 75% identity, more preferably about 85% identity to the target polynucleotide; with greater than about 90% identity to target-polynucleotide being especially preferred. Preferably, moderately stringent conditions are conditions equivalent to hybridization in 50% formamide, 5× Denhart's solution, 5× SSPE, 0.2% SDS at 42° C., followed by washing in 0.2×SSPE, 0.2% SDS, at 65° C.

High stringency hybridization refers to conditions that permit hybridization of only those polynucleotide sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in 50% formamide, 5× Denhart's solution, 5×SSPE, 0.2% SDS at 42° C., followed by washing in 0.1×SSPE, and 0.1% SDS at 65° C.

Low stringency hybridization refers to conditions equivalent to hybridization in 10% formamide, 5× Denhart's solution, 6×SSPE, 0.2% SDS at 42° C., followed by washing in 1×SSPE, 0.2% SDS, at 50° C. Denhart's solution and SSPE (see, e.g., Sambrook et al., Molecular Cloning, A Laboratory Manual, Cold Spring Harbor Laboratory Press, 1989) are well known to those of skill in the art as are other suitable hybridization buffers.

Polypeptides or polynucleotides encoding polypeptides that are substantially the same can also be identified as those having minor sequence modifications with respect to each other, so long as the polypeptides have similar functional activities. Two or more polynucleotides or the polypeptides encoded therefrom can have a variety of similar activities including, for example, immunogenicity, antigenicity, enzymatic activity, binding activity, or other biological property, including invertebrate foraging behavior-modulating activity.

A modification of a polynucleotide molecule can also include substitutions that do not change the encoded amino acid sequence due to the degeneracy of the genetic code. Such modifications can correspond to variations that are made deliberately, or which occur as mutations during polynucleotide replication. Additionally, a modification of a polynucleotide molecule can correspond to a splice variant form of the recited sequence.

Additionally, a fragment of a mammalian polynucleotide can be substantially the same as an invertebrate polynucleotide or a fragment of an invertebrate polynucleotide. A mammalian polynucleotide can be substantially the same as an invertebrate polynucleotide, for example, when one of several domains encoded by a mammalian polynucleotide corresponds to a domain encoded by an invertebrate protein. Such a fragment typically is encoded by at least 30 nucleotides, and the mammalian and invertebrate polynucleotides encoding that fragment share at least about 50% identity, at least about 60% identity, at least about 70% identity, at least about 80% identity, at least about 90% identity, at least about 95% identity or at least about 98% identity. Methods for determining that a fragment of a mammalian polynucleotide is substantially the same as an invertebrate polynucleotide or a fragment of an invertebrate polynucleotide include those described above for comparing mammalian and invertebrate polynucleotides. Such a fragment can be encoded by 30 or more nucleotides, for example, 45 or more nucleotides, 60 or more nucleotides, 90 or more nucleotides, 150 or more nucleotides, 210 or more nucleotides, or 300 or more nucleotides.

Biological functions retained by a fragment can include the ability to modulate ADHD, hypertension or a disease associated with a NO/cGMP-dependent kinase network in a mammal, the ability to modulate invertebrate foraging, the ability to bind an antibody that binds to a full-length protein from which the fragment was derived, or an enzymatic or binding activity characteristic of the full length protein. For example, peptides corresponding to 30 amino acid domains of CaMKII and PKC inhibit the respective full length enzymes as described in Kane et al., Neuron 18:307-314 (1997), Broughton et al., J. Cell. Biochem. 62:484-494 (1996), Broughton et al., J. Cell. Biochem. 60:584-600 (1996) and Griffith et al., Neuron 10:501-509 (1993).

Methods for determining that two sequences are substantially the same are well known in the art. For example, one method for determining if two sequences are substantially the same is BLAST, Basic Local Alignment Search Tool, which can be used according to default parameters as described by Tatiana et al., FEMS Microbial Lett. 174:247-250 (1999) or on the National Center for Biotechnology Information web page at ncbi.nlm.gov/BLAST/. BLAST is a set of similarity search programs designed to examine all available sequence databases and can function to search for similarities in amino acid or nucleic acid sequences. A BLAST search provides search scores that have a well-defined statistical interpretation. Furthermore, BLAST uses a heuristic algorithm that seeks local alignments and is therefore able to detect relationships among sequences which share only isolated regions of similarity including, for example, protein domains (Altschul et al., J. Mol. Biol. 215:403-410 (1990)).

In addition to the originally described BLAST (Altschul et al., supra, 1990), modifications to the algorithm have been made (Altschul et al., Nucleic Acids Res. 25:3389-3402 (1997)). One modification is Gapped BLAST, which allows gaps, either insertions or deletions, to be introduced into alignments. Allowing gaps in alignments tends to reflect biologic relationships more closely. For example, gapped BLAST can be used to identify sequence identity within similar domains of two or more proteins. A second modification is PSI-BLAST, which is a sensitive way to search for sequence homologs. PSI-BLAST performs an initial Gapped BLAST search and uses information from any significant alignments to construct a position-specific score matrix, which replaces the query sequence for the next round of database searching. A PSI-BLAST search is often more sensitive to weak but biologically relevant sequence similarities.

A second resource that can be used to determine if two sequences are substantially the same is PROSITE, available on the world wide web at ExPASy. PROSITE is a method of determining the function of uncharacterized proteins translated from genomic or cDNA sequences (Bairoch et al., Nucleic Acids Res. 25:217-221 (1997)). PROSITE consists of a database of biologically significant sites and patterns that can be used to identify which known family of proteins, if any, the new sequence belongs. In some cases, the sequence of an unknown protein is too distantly related to any protein of known structure to detect similarity by overall sequence alignment. However, a protein that is substantially the same as another protein can be identified by the occurrence in its sequence of a particular cluster of amino acid residues, which can be called a pattern, motif, signature or fingerprint, that is substantially the same as a particular cluster of amino acid residues in the other protein including, for example, those found in similar domains. PROSITE uses a computer algorithm to search for motifs that identify proteins as family members. PROSITE also maintains a compilation of previously identified motifs, which can be used to determine if a newly identified protein is a member of a known protein family.

Therefore, the invention further provides a method of identifying a polypeptide involved in a disease associated with a NO/cGMP-dependent kinase network in a mammal. The method consists of (a) obtaining a first and a second strain of an invertebrate; (b) subjecting the first and second invertebrate strains to conditions in which the first strain exhibits a foraging behavior different than a foraging behavior exhibited by the second strain; (c) measuring polynucleotide expression levels in the first and second strains; (d) determining the amino acid sequence of a polypeptide encoded by one or more polynucleotides; and (e) identifying one or more polypeptides encoded by the one or more polynucleotides that are differentially expressed in the first strain relative to the second strain, whereby a mammalian polypeptide having substantially the same amino acid sequence as the one or more differentially expressed polypeptides is involved in a disease associated with a NO/cGMP-dependent kinase network in a mammal.

Therefore, the invention provides a method of identifying a polypeptide involved in ADHD or hypertension in a mammal. The method consists of (a) obtaining a first and a second strain of an invertebrate; (b) subjecting the first and second invertebrate strains to conditions in which the first strain exhibits a foraging behavior different than a foraging behavior exhibited by the second strain; (c) measuring polynucleotide expression levels in the first and second strains; (d) determining the amino acid sequence of a polypeptide encoded by one or more polynucleotides; and (e) identifying one or more polypeptides encoded by the one or more polynucleotides that are differentially expressed in the first strain relative to the second strain, whereby a mammalian polypeptide having substantially the same amino acid sequence as the one or more differentially expressed polypeptides is involved in ADHD or hypertension in a mammal.

The invention provides an isolated polynucleotide, or fragment thereof, having ADHD-altering activity in a mammal and having substantially the same nucleic acid sequence as a polynucleotide selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104. The invention also provides an isolated polynucleotide, or fragment thereof, having hypertension-altering activity in a mammal and having substantially the same nucleic acid sequence as a polynucleotide selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104.

Genetic methods of identifying new genes associated with invertebrate foraging behavior that are applicable to a variety of invertebrates are known in the art. For example, the invertebrate can be mutagenized using chemicals, radiation or insertions (e.g. transposons, such as P element mutagenesis), appropriate crosses performed, and the progeny screened for phenotypic differences in foraging behavior compared with normal controls. The gene can then be identified by a variety of methods including, for example, linkage analysis or rescue of the gene targeted by the inserted element. Genetic methods of identifying genes are described for Drosophila, for example, in Greenspan, Fly Pushing: The Theory and Practice of Drosophila Genetics, Cold Spring Harbor Laboratory Press (1997).

There are numerous important diagnostic, therapeutic, and screening applications that arise from identification of novel genes that modulate ADHD, hypertension or other diseases associated with a NO/cGMP-dependent kinase network in a mammal. For example, an expression or activity profile of one or many genes that modulate a disease can be established as a molecular fingerprint of the presence or severity of the disease. Thus, in diagnostic applications, it can readily be determined, by comparing the expression profile of an individual to one or more reference profiles, whether that individual suffers from, or is susceptible to, a particular disease. Likewise, the sensitivity of a NO/cGMP-dependent kinase network in a mammal and the effect of medications or medical procedures on the network in a mammal, can be determined at the molecular level. Such determinations allow for more appropriate determination and use of therapeutics for treating disorders such as ADHD or hypertension.

In screening applications, identification of genes that modulate ADHD, hypertension or other diseases associated with a NO/cGMP-dependent kinase network in a mammal allows novel compounds to be identified, lead compounds to be validated, and the molecular effects of these compounds and other known compounds to be characterized, by determining the effect of these compounds on an expression profile. For example, the ability of a compound, administered to an individual with a particular disorder, to alter the expression profile to correspond more closely to the profile of an unaffected or normal individual can be determined as described herein.

An isolated polynucleotide molecule of the invention that modulates ADHD, hypertension or other diseases associated with a NO/cGMP-dependent kinase network in a mammal can contain a sequence that is substantially the same as a sequence from a polynucleotide that is differentially expressed in invertebrate having different foraging behaviors. As described in Example II, SEQ ID NOS: 50-56 and SEQ ID NO: 77 correspond to polynucleotides having increased expression in Rovers as compared to sitters. In addition, SEQ ID NO: 77, SEQ ID NO: 79, and SEQ ID NO: 85 correspond to polynucleotides encoding polypeptides that are expressed at higher levels in Rovers as compared to sitters. Polypeptides that are expressed at higher levels in Rovers as compared to sitters include, for example, SEQ ID NO: 78, SEQ ID NO: 80, SEQ ID NO: 81, SEQ ID NO: 83 and SEQ ID NO: 86. As further described in Example II, SEQ ID NOS: 57-59 correspond to polynucleotides having decreased expression in Rovers as compared to sitters. In addition, SEQ ID NO: 75, SEQ ID NO: 87 and SEQ ID NO: 89 correspond to polynucleotides encoding polypeptides that are expressed at lower levels in Rovers as compared to sitters. Polypeptides that are expressed at lower levels in Rovers as compared to sitters include, for example, SEQ ID NO: 76, SEQ ID NO: 88 and SEQ ID NO: 90.

In accordance with the present invention, various polynucleotides identified by the methods of the invention are homologous to known mammalian polynucleotides including, for example, SEQ ID NOS: 47-61, SEQ ID NO: 75, SEQ ID NO: 77, SEQ ID NO: 79, SEQ ID NO: 85, SEQ ID NO: 87, and SEQ ID NO: 89 which are homologous to SEQ ID NOS: 62-74, SEQ ID NO: 91, SEQ ID NO: 93, SEQ ID NO: 95, SEQ ID NO: 100, SEQ ID NO: 102, and SEQ ID NO: 104 respectively as shown in Table 1. These polynucleotides and the polypeptides they encode are known in the art. One skilled in the art will be able to make and use these polynucleotides and their respective polypeptide products in the methods of the invention according to their known properties as described for example in the Genbank or flybase databases. Table 1 provides database accession numbers for these sequences.

TABLE 1

| | Fly Homolog | | | Human Homolog | |
|---|---|---|---|---|---|
| gene | SEQ ID NO | Genbank | flybase | SEQ ID NO | Genbank |
| foraging/dg2 | 47 | M30413 | | 62 | Z92869 |
| alcohol | 75 | M17837 | FBgn0000055 | 91 | NM_000667 |
| dehydrogenase | | | | | |
| inositol | 48 | AF069513 | FBgn0016672 | 63 | NM_002194 |
| polyphosphate1 1- | | | | | |
| phosphatase | | | | | |
| inositol 1,4,5- | 49 | Z18535 | FBgn0010051 | 64 | L38019 |
| tri-phosphate | | | | | |
| receptor | | | | | |
| Dead Box-1 | 50 | AF057167 | FBgn0015075 | 65 | NM_004939 |
| CNS-specific | 51 | AF077736 | FBgn0026197 | 66 | HS321I20 |
| protein Noe | | | | | |
| cellular repressor | 77 | AF084522 | FBgn0025456 | 93 | NM_003851 |
| of E1A-stimulated | | | | | |
| genes | | | | | |
| 14-3-3 ε | 52 | U84898 | FBgn0020238 | 67 | U54778 |
| casein kinase II α | 53 | M16534 | FBgn0000258 | 68 | J02853 |
| subunit | | | | | |
| mRNA sequence | 54 | AF007164 | | 69 | U12918 |
| similar to | | | | | |
| syntaxin I | | | | | |
| ADP/ATP | 55 | AI405031 | FBgn0003360 | 70 | NM_001151 |
| translocase/sesB | | | | | |
| mitochondrial | 56 | AJ000880 | FBgn0004363 | 71 | L06132 |
| porin | | | | | |
| neuron specific | 57 | U36477 | FBgn0004880 | 72 | NM_003425 |
| zinc finger | | | | | |
| transcription | | | | | |
| factor (scratch) | | | | | |
| ectozoan-regulated | 58 | U25686 | FBgn0013948 | | |
| (E93) | | | | | |
| centrosomal and | 59 | AI544153 | | | |
| chromosomal factor | | | | | |
| (ccf) | | | | | |
| activin β | 79 | AF054822 | FBgn0024913 | 95 | A14422 |
| precursor | | | | | |
| dynamic-like | | S17974 | | | A40671 |
| paramyosin | | P35416 | | | P11055 |
| mitochondrial ATP | 85 | Y07894 | | 100 | NM_004046 |
| synthase α subunit | | | | | |
| Fas-associated | 87 | AB013610 | FBgn0025608 | 102 | KIAA0942 |
| factor (FFAF) | | | | | |
| lamin precursor | 89 | X07278 | FBgn0002525 | 104 | NM_005572 |
| 18S, 5.8S, 2S and | 60 | 21017 | FBgn0016760 | 73 | U13369 |
| 28S rRNA genes | | | | | |
| ribosomal protein | 61 | DRORBS6 | FBgn0004922 | 74 | NM_001010 |
| S6 gene | | | | | |

The isolated polynucleotide molecules comprising SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 or SEQ ID NO: 104. can hybridize to mammalian polynucleotides, and thus can be used in the diagnostic and screening methods described herein. Additionally, the isolated polynucleotide molecules containing sequences substantially the same as one of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 or SEQ ID NO: 104. can be administered in gene therapy methods including, for example, antisense methods, to decrease expression of polypeptides that modulate ADHD, hypertension or other disease associated with a NO/cGMP-dependent kinase network in a mammal. The isolated polynucleotide molecules of the invention can also be used as probes or primers to identify larger cDNAs or genomic DNA, or to identify homologs of the polynucleotide molecules in other species. The isolated polynucleotide molecules can further be expressed to produce polypeptides for use in producing antibodies or for designing or identifying inhibitory or stimulatory compounds. It is understood that the isolated polynucleotide molecules of the invention can be used for a variety of other uses known to those skilled in the art.

The invention also provides isolated polynucleotides containing at least 15 contiguous nucleotides of a nucleotide sequence referenced as SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102, SEQ ID NO: 104, or an antisense strand thereof. An polynucleotide of the invention can include, for example, at least 15 contiguous nucleotides from the reference nucleotide sequence, can include at least 16, 17, 18, 19, 20 or at least 25 contiguous nucleotides, and often includes at least 30, 40, 50, 60, 70, 80, 90, 100, 125, 150, 175, 200 or more contiguous nucleotides from the reference nucleotide sequence. The isolated polynucleotides of the invention are able to specifically hybridize to polynucleotide molecules associated with invertebrate foraging or with modulation of ADHD, hypertension or other disease associated with a NO/cGMP-dependent kinase network in a mammal, under moderately or highly stringent hybridization conditions and thus can be advantageously used, for example, as probes in a diagnostic assay; as sequencing or PCR primers; as antisense reagents to administer to an individual to block gene expression; or in other applications known to those skilled in the art in which hybridization to a polynucleotide molecule is desirable.

In one embodiment, the invention provides a primer pair for detecting polynucleotide molecules associated with invertebrate foraging or with ADHD, hypertension or other disease associated with a NO/cGMP-dependent kinase network in a mammal. The primer pair contains two isolated polynucleotides, each containing at least 15 contiguous nucleotides of one of the nucleotide sequences referenced as SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104., with one sequence annealing to the sense strand, and one sequence annealing to the anti-sense strand. The primer pair can be used, for example, to amplify polynucleotide molecules associated with invertebrate foraging or with modulation of a mammalian vestibular system by RT-PCR or PCR.

The isolated polynucleotide molecules of the invention can be produced or isolated by methods known in the art. The method chosen will depend, for example, on the type of polynucleotide molecule one intends to isolate. Those skilled in the art, based on knowledge of the nucleotide sequences disclosed herein, can readily isolate the polynucleotide molecules of the invention as genomic DNA, or desired introns, exons or regulatory sequences therefrom; as full-length cDNA or desired fragments therefrom; or as full-length mRNA or desired fragments therefrom, by methods known in the art.

A useful method for producing an isolated polynucleotide molecule of the invention involves, for example, amplification of the polynucleotide molecule using the methods such as the polymerase chain reaction (PCR) with polynucleotide primers specific for the desired polynucleotide molecule. Amplification procedures such as PCR or reverse-transcription PCR (RT-PCR) can be used to produce a polynucleotide molecule having any desired nucleotide boundaries. Desired modifications to the nucleic acid sequence can also be introduced by choosing an appropriate primer with one or more additions, deletions or substitutions. Such polynucleotide molecules can be amplified exponentially starting from as little as a single gene or mRNA copy, from any cell, tissue or species of interest.

A further method of producing an isolated polynucleotide molecule of the invention is by screening a library, such as a genomic DNA library, cDNA library or expression library, with a detectable agent. Such libraries are commercially available or can be produced from any desired tissue, cell, or species of interest using methods known in the art. For example, a cDNA or genomic library can be screened by hybridization with a detectably labeled polynucleotide molecule having a nucleotide sequence disclosed herein. Additionally, an expression library can be screened with an antibody raised against a polypeptide encoded by a polynucleotide disclosed herein. The library clones containing polynucleotide molecules of the invention can be isolated from other clones by methods known in the art and, if desired, fragments therefrom can be isolated by restriction enzyme digestion and gel electrophoresis.

Furthermore, isolated polynucleotide molecules of the invention can be produced by synthetic means. For example, a single strand of a polynucleotide molecule can be chemically synthesized by automated synthesis methods known in the art. The complementary strand can likewise be synthesized and a double-stranded molecule made by annealing the complementary strands. Direct synthesis is particularly advantageous for producing relatively short molecules, such as polynucleotide probes and primers, and polynucleotide molecules containing modified nucleotides or linkages. However, overlapping strands with complementary overhanging regions can be synthesized and annealed to create double stranded polynucleotides that are longer than those that can be efficiently synthesized as a single strand.

In one embodiment, the isolated polynucleotide molecules of the invention are attached to a solid support, such as a chip, filter, glass slide or culture plate, by either covalent or non-covalent methods. Methods of attaching polynucleotide molecules to a solid support, and the uses of polynucleotides in this format in a variety of assays, including manual and automated hybridization assays, are well known in the art. A solid support format is particularly appropriate for automated diagnostic or screening methods, where simultaneous hybridization to a large number of polynucleotides associated with invertebrate foraging or with modulation of ADHD, hypertension or other disease associated with a NO/cGMP-dependent kinase network in a mammal is desired, or when a large number of samples are being handled.

In another embodiment, the invention provides kits containing two or more isolated polynucleotide molecules. At least one polynucleotide molecule of the kit contains a nucleotide sequence selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104., or minor modification thereof or at least 15 contiguous nucleotides of a nucleic acid sequence referenced as SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104. An exemplary kit is a solid support containing an array of isolated polynucleotide molecules of the invention, including, for example, at least 3, 5, 10, 20, 30, 40, 50, 75, 100, 265 or more isolated polynucleotide molecules.

A further exemplary kit contains one or more PCR primer pairs, or two or more hybridization probes, which optionally can be labeled with a detectable moiety for detection of polynucleotide molecules. The kits of the invention can additionally contain instructions for use of the molecules for diagnostic purposes in a clinical setting, or for drug screening purposes in a laboratory setting.

If desired, the kits containing two or more isolated polynucleotide molecules can contain polynucleotide molecules corresponding to genes that are up regulated in invertebrates exhibiting negative foraging behavior, or are down regulated in invertebrates exhibiting negative foraging behavior. Additionally, the kits containing two or more isolated polynucleotide molecules can contain polynucleotide molecules corresponding to sequences identified from Drosophila screens or other invertebrate screens, from rat screens, from screens in other mammals, or any combination thereof.

The invention also provides a vector containing an isolated polynucleotide molecule associated with invertebrate foraging or with modulation of ADHD, hypertension or other disease associated with a NO/cGMP-dependent kinase network in a mammal. The vectors of the invention are useful, for example, for cloning and amplifying an isolated polynucleotide molecule, for recombinantly expressing a polypeptide, or in gene therapy applications.

Suitable expression vectors are well-known in the art and include vectors capable of expressing a polynucleotide operatively linked to a regulatory sequence or element such as a promoter region or enhancer region that is capable of regulating expression. Promoters or enhancers, depending upon the nature of the regulation, can be constitutive or inducible. The regulatory sequences or regulatory elements are operatively linked to a polynucleotide of the invention in an appropriate orientation to allow transcription of the polynucleotide.

Appropriate expression vectors include those that are replicable in eukaryotic cells and/or prokaryotic cells and those that remain episomal or those which integrate into the host cell genome. Suitable vectors for expression in prokaryotic or eukaryotic cells are well known to those skilled in the art as described, for example, in Ausubel et al., Current Protocols in Molecular Biology, John Wiley and Sons, Baltimore, Md. (2000). Vectors useful for expression in eukaryotic cells can include, for example, regulatory elements including the SV40 early promoter, the cytomegalovirus (CMV) promoter, the mouse mammary tumor virus (MMTV) steroid-inducible promoter, Moloney murine leukemia virus (MMLV) promoter, and the like. A vector useful in the methods of the invention can include, for example, viral vectors such as a bacteriophage, a baculovirus or a retro virus; cosmids or plasmids; and, particularly for cloning large polynucleotide molecules, bacterial artificial chromosome vectors (BACs) and yeast artificial chromosome vectors (YACs). Such vectors are commercially available, and their uses are well known in the art. One skilled in the art will know or can readily determine an appropriate promoter for expression in a particular host cell.

Appropriate host cells, include for example, bacteria and corresponding bacteriophage expression systems, yeast, avian, insect and mammalian cells and compatible expression systems known in the art corresponding to each host species. Methods for isolating, cloning and expressing polynucleotide molecules are well known in the art and are described, for example, in Sambrook et al., supra and in Ausubel et al., supra. The choice of a particular vector and host system for expression can be determined by those skilled in the art and will depend on the preference of the user.

Recombinant cells can be generated by introducing into a host cell a vector or population of vectors containing a polynucleotide molecule encoding a binding polypeptide. A recombinant cell can be produced by transducing, transecting or other means of introducing genetic material using a variety of methods known in the art to incorporate exogenous polynucleotides into a cell or its genome. Exemplary host cells that can be used include mammalian primary cells; established mammalian cell lines, such as COS, CHO, HeLa, NIH3T3, HEK 293 and PC12 cells; amphibian cells, such as Xenopus embryos and oocytes; and other vertebrate cells. Exemplary host cells also include insect cells such as Drosophila, yeast cells such as Saccharomyces cerevisiae, Saccharomyces ombe, or Pichia pastoris, and prokaryotic cells such as Escherichia coli.

In one embodiment, a polynucleotide of the invention can be delivered into mammalian cells, either in vivo or in vitro using suitable vectors well-known in the art. Suitable vectors for delivering a polynucleotide encoding a polypeptide to a mammalian cell, include viral vectors such as retro viral vectors, adenovirus, adeno-associated virus, lentivirus, herpes virus, as well as non-viral vectors such as plasmid vectors.

Viral based systems provide the advantage of being able to introduce relatively high levels of a heterologous polynucleotide into a variety of cells. Suitable viral vectors for introducing a polynucleotide encoding a polypeptide into mammalian cells are well known in the art. These viral vectors include, for example, Herpes simplex virus vectors (Geller et al., Science, 241:1667-1669 (1988)); vaccinia virus vectors (Piccini et al., Meth. Enzymology, 153:545-563 (1987)); cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84)); Moloney murine leukemia virus vectors (Danos et al., Proc. Natl. Acad. Sci. USA, 85:6460-6464 (1988); Blaese et al., Science, 270:475-479 (1995); Onodera et al., J. Virol., 72:1769-1774 (1998)); adenovirus vectors (Berkner, Biotechniques, 6:616-626 (1988); Cotten et al., Proc. Natl. Acad. Sci. USA, 89:6094-6098 (1992); Graham et al., Meth. Mol. Biol., 7:109-127 (1991); Li et al., Human Gene Therapy, 4:403-409 (1993); Zabner et al., Nature Genetics, 6:75-83 (1994)); adeno-associated virus vectors (Goldman et al., Human Gene Therapy, 10:2261-2268 (1997); Greelish et al., Nature Med., 5:439-443 (1999); Wang et al., Proc. Natl. Acad. Sci. USA, 96:3906-3910 (1999); Snyder et al., Nature Med., 5:64-70 (1999); Herzog et al., Nature Med., 5:56-63 (1999)); retro virus vectors (Donahue et al., Nature Med., 4:181-186 (1998); Shackleford et al., Proc. Natl. Acad. Sci. USA, 85:9655-9659 (1988); U.S. Pat. Nos. 4,405,712, 4,650,764 and 5,252,479, and WIPO publications WO 92/07573, WO 90/06997, WO 89/05345, WO 92/05266 and WO 92/14829; and lentivirus vectors (Kafri et al., Nature Genetics, 17:314-317 (1997)).

The invention further provides transgenic non-human animals that are capable of expressing wild-type polynucleotides, dominant-negative polynucleotides, antisense polynucleotides, or ribozymes that target polynucleotides, where the polynucleotides are associated with invertebrate foraging or with modulation of ADHD, hypertension or other disease associated with a NO/cGMP-dependent kinase network in a mammal. Such animals have correspondingly altered expression of polypeptides associated with invertebrate foraging or with modulation of ADHD, hypertension or other disease associated with a NO/cGMP-dependent kinase network in a mammal, and can thus be used to elucidate or confirm the function of such polypeptides, or in whole-animal assays to determine or validate the physiological effect of compounds that potentially modulate ADHD, hypertension or other disease associated with a NO/cGMP-dependent kinase network in a mammal. The transgene may additionally comprise an inducible promoter and/or a tissue specific regulatory element, so that expression can be induced or restricted to specific cell types. Exemplary transgenic non-human animals expressing polynucleotides and polynucleotides that alter gene expression include mouse and Drosophila. Methods of producing transgenic animals are well known in the art.

The present invention provides an isolated polypeptide having ADHD-altering activity in a mammal, or fragment thereof, having substantially the same amino acid sequence as an amino acid sequence encoded by a polynucleotide sequence selected from the group consisting of SEQ ID NOS: 1-75, SEQ ID NO: 77, SEQ ID NO: 79, SEQ ID NO: 85, SEQ ID NO: 87, SEQ ID NO:89, SEQ ID NO: 91, SEQ ID NO: 93, SEQ ID NO: 95, SEQ ID NO: 100, SEQ ID NO: 102 or SEQ ID NO: 104. The invention further provides an isolated polypeptide having hypertension-altering activity in a mammal, or fragment thereof, having substantially the same amino acid sequence as an amino acid sequence encoded by a nucleic acid sequence selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 or SEQ ID NO: 104. The invention also provides an isolated polypeptide having ADHD-altering activity or hypertension-altering activity in a mammal, or fragment thereof, comprising a polypeptide having a molecular weight of 50 kD and pI of 4.1, a molecular weight of 28 kD and pI of 8.7, a molecular weight of 36 kD and pI of 6.0, a molecular weight of 34 kD and pI of 6.3, a molecular weight of 25 kD and pI of 5.9, a molecular weight of 12 kD and pI of 5.7, a molecular weight of 12 kD and pI of 6.4, a molecular weight of 12 kD and pI of 6.4, or a molecular weight of 29 kD and pI of 6.5, wherein the molecular weight and isoelectric point (pI) are determined by 2 dimensional polyacrylamide gel electrophoresis using the methods described in Unlu et al., Electrophoresis 18:2071 (1997) in tandem with excision of polypeptide containing bands from other bands in the gel, in gel trypsin digestion, extraction, purification and analysis by MALDI-TOF as described in Helman et al. Anal. Biochem. 224:451 (1995). One skilled in the art will know that the above described molecular weights and isoelectric points, being determined from two dimensional polyacrylamide gel electrophoresis, can differ considerably from those predicted based on sequence data alone. Identification and characterization of polypeptides having the above described features is further described in Example II.

Isolated polypeptides of the invention can be used in a variety of applications. For example, isolated polypeptides can be used to generate specific antibodies, or in screening or validation methods where it is desired to identify or characterize compounds that alter the activity of polypeptides that with modulate a mammalian vestibular system.

The isolated polypeptides of the invention can be prepared by methods known in the art, including biochemical, recombinant and synthetic methods. For example, invention polypeptides can be purified by routine biochemical methods from neural cells or other cells that express abundant amounts of the polypeptide. Methods for isolating polypeptides are well known in the art as described, for example, in Scopes, Protein Purification: Principles and Practice, 3^{rd} Ed., Springer-Verlag, New York (1994); Duetscher, Methods in Enzymology, Vol 182, Academic Press, San Diego (1990), and Coligan et al., Current protocols in Protein Science, John Wiley and Sons, Baltimore, Md. (2000).

An invention polypeptide can also be produced by recombinant methods as described above. Recombinant methods involve expressing a polynucleotide molecule encoding the desired polypeptide in a host cell or cell extract, and isolating the recombinant polypeptide, such as by routine biochemical purification methods also described above. Methods for producing and expressing recombinant polypeptides in vitro and in prokaryotic and eukaryotic host cells are well known in the art as described, for example, in Goeddel, Methods in Enzymology, Vol 185, Academic Press, San Diego (1990); Wu, Methods in Enzymology, Vol 217, Academic Press, San Diego (1993); Sambrook et al., supra, and in Ausebel et al., supra. Furthermore, invention polypeptides can be produced by synthetic methods well known in the art including, for example, Merrifield solid phase synthesis, t-Boc based synthesis, Fmoc synthesis and variations thereof.

A polypeptide of the invention can accommodate minor modifications that can confer additional properties onto the polypeptide so long as such modifications do not inhibit the polypeptides activity as it relates to invertebrate foraging or modulation of ADHD, hypertension or other disease associated with a NO/cGMP-dependent kinase network in a mammal. A modification can be made, for example, to facilitate identification and purification of the recombinant polypeptide. In this regard, it is often desirable to insert or add, in-frame with the coding sequence, polynucleotides that encode epitome tags or other binding sequences, or sequences that direct secretion of the polypeptide. A binding sequence that can be used to capture a polypeptide includes, for example, a biotinylation sequence, polyhistidine tag (Qiagen; Chatsworth, Calif.), antibody epitome such as the flag peptide (Sigma; St Louis, Mo.), glutathione-S-transferase (Amersham Pharmacia; Piscataway, N.J.), cellulose binding domain (Novagen; Madison, Wis.), calmodulin (Stratagene; San Diego, Calif.), staphylococcus protein A (Pharmacia; Uppsala, Sweden), maltose binding protein (New England BioLabs; Beverley, Mass.) or strep-tag (Genosys; Woodlands, Tex.) or minor modifications thereof. A modification can also be made to increase stability and can include, for example, incorporation of a cysteine to form a thioether cross-link, removal of a protease recognition sequence, addition of a charged amino acid to promote ionic interactions, or addition of a hydrophobic amino acid to promote hydrophobic interactions. In addition a polypeptide of the invention can be modified to incorporate additional amino acids, remove amino acids, substitute amino acids, chemically modified amino acids or incorporate non-natural amino acids.

An antibody specific for an isolated polypeptide of the invention is also provided. An antibody specific for a polypeptide of the invention can be a polyclonal or monoclonal antibody. Such antibodies can be used, for example, in diagnostic assays such as ELISA assays to detect or quantitate the expression of polypeptides of the invention; to purify polypeptides of the invention; or as therapeutic compounds to selectively target polypeptide of the invention. Such antibodies, if desired, can be bound to a solid support, such as a chip, filter, glass slide or culture plate. An antibody of the invention can be prepared and used according to methods that are well known in the art as described, for example, in Harlow and Lane, supra.

The invention provides diagnostic methods based on the newly identified and characterized polynucleotides described herein. In one embodiment, the invention provides a method of diagnosing ADHD, hypertension or other disease associated with a NO/cGMP-dependent kinase network in a mammal. The method consists of determining an expression profile of the individual, and comparing that profile to a reference profile indicative of the particular disease. Correspondence between the profile of the individual and the reference profile indicates that the individual has the disorder. In one embodiment, at least one of the polynucleotides profiled is a polynucleotide containing a nucleic acid sequence substantially the same as one of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 or SEQ ID NO: 104. Typically, at least one of the polynucleotides profiled is selected from the group consisting of foraging/dg2; alcohol dehydrogenase (SEQ ID NO: 76); inositol polyphosphatel 1-phosphatase; inositol 1,4,5-tri-phosphate receptor; Dead Box-1; CNS-specific protein Noe; cellular repressor of E1A-stimulated genes (SEQ ID NO: 78); 14-3-3 ε; casein kinase II α subunit; syntaxin I; ADP/ATP translocase/sesB ;mitochondrial porin; neuron specific zinc finger transcription factor (scratch); ectozoan-regulated (E93); centrosomal and chromosomal factor (ccf); activin β precursor (SEQ ID NO: 80); dynamic-like (SEQ ID NO: 81); paramyosin (SEQ ID NO: 83); mitochondrial ATP synthase α subunit (SEQ ID NO: 86); Fas-associated factor (FFAF) (SEQ ID NO: 88); lamin precursor (SEQ ID NO: 90); and ribosomal protein S6.

The methods of diagnosing ADHD, hypertension or other disease associated with a NO/cGMP-dependent kinase network in a mammal have numerous applications. Appropriate diagnosis of a such diseases will allow more effective treatments: using currently available treatments for ADHD, hypertension or other disease associated with a NO/cGMP-dependent kinase network in a mammal; using compounds identified from the screens described herein; using the therapeutic methods described herein; or any combination of these treatments. Likewise, methods of diagnosing ADHD, hypertension or other disease associated with a NO/cGMP-dependent kinase network in a mammal are applicable to monitoring the course of therapy for the disorder, such that appropriate modifications can be made if needed.

Furthermore, the methods of diagnosing ADHD, hypertension or other disease associated with a NO/cGMP-dependent kinase network in a mammal are applicable to screening for such diseases among the general population, or among populations in whom such diseases influence the safety of the individual or the general population. The diagnostic methods of the invention can also advantageously be used to characterize a previously unrecognized disease associated with a NO/cGMP-dependent kinase network in a mammal, or to newly categorize such a disease, based on characteristic patterns of expression or activity of polynucleotides associated with invertebrate foraging. Re-categorization of a disease can lead to new or alternate treatments to increase efficacy, reduce side effects or otherwise tailor treatment to the needs of an individual. The diagnostic methods of the invention can also be advantageously used to identify the specific polynucleotides most closely associated with, and thus likely to play a causative role, in ADHD, hypertension or other disease associated with a NO/cGMP-dependent kinase network in a mammal.

Those skilled in the art understand that the methods described herein for diagnosing ADHD, hypertension or other disease associated with a NO/cGMP-dependent kinase network in a mammal can readily be applied to methods of screening for novel compounds; to methods of validating the efficacy of compounds identified by other methods to modulate ADHD, hypertension or other disease associated with a NO/cGMP-dependent kinase network in a mammal; to methods of determining effective dose, time and route of administration of known therapeutic compounds; to methods of determining the molecular mechanisms of action of known therapeutic compounds; and the like. Such methods can be performed in laboratory animals, such as mice, rats, rabbits, dogs, cats, pigs or primates, or in a clinical setting with humans.

The invention provides a method of modulating ADHD, hypertension or other disease associated with an NO/cGMP-dependent protein kinase network by administering to a mammalian subject an effective amount of a compound that modulates the expression of a polynucleotide selected from the group consisting of foraging/dg2 (SEQ ID NO: 47); alcohol dehydrogenase (SEQ ID NO: 75); inositol polyphosphatel 1-phosphatase (SEQ ID NO: 48); inositol 1,4,5-tri-phosphate receptor (SEQ ID NO: 49); Dead Box-1 (SEQ ID NO: 50); CNS-specific protein Noe (SEQ ID NO: 51); cellular repressor of E1A-stimulated genes (SEQ ID NO: 77); 14-3-3 ε (SEQ ID NO: 52); casein kinase II α subunit (SEQ ID NO: 53); mRNA sequence similar to syntaxin I (SEQ ID NO: 54); ADP/ATP translocase/sesB (SEQ ID NO: 55); mitochondrial porin (SEQ ID NO: 56); neuron specific zinc finger transcription factor (scratch) (SEQ ID NO: 57); ectozoan-regulated (E93)(SEQ ID NO: 58); centrosomal and chromosomal factor (ccf) (SEQ ID NO: 59); activin β precursor (SEQ ID NO: 79); dynamic-like (SEQ ID NO: 81); paramyosin (SEQ ID NO: 83); mitochondrial ATP synthase a subunit (SEQ ID NO: 85); Fas-associated factor (FFAF)(SEQ ID NO: 87); lamin precursor (SEQ ID NO: 89); 18S, 5.8S, 2S and 28S rRNA genes (SEQ ID NO: 60); and ribosomal protein S6 gene (SEQ ID NO: 61).

The invention also provides a method of modulating ADHD, hypertension or other disease associated with an NO/cGMP-dependent protein kinase network by administering to a mammalian subject an effective amount of a compound that modulates the activity or expression of a polypeptide selected from the group consisting of foraging/dg2; alcohol dehydrogenase (SEQ ID NO: 76); inositol polyphosphatel 1-phosphatase; inositol 1,4,5-tri-phosphate receptor; Dead Box-1; CNS-specific protein Noe; cellular repressor of ElA-stimulated genes (SEQ ID NO: 78); 14-3-3 ε; casein kinase II α subunit; syntaxin I; ADP/ATP translocase/sesB ; mitochondrial porin; neuron specific zinc finger transcription factor (scratch); ectozoan-regulated (E93); centrosomal and chromosomal factor (ccf); activin β precursor (SEQ ID NO: 80); dynamic-like (SEQ ID NO: 81); paramyosin (SEQ ID NO: 83); mitochondrial ATP synthase α subunit (SEQ ID NO: 86); Fas-associated factor (FFAF)(SEQ ID NO: 88); lamin precursor (SEQ ID NO: 90); and ribosomal protein S6.

In addition, the invention provides a method of modulating ADHD, hypertension or other disease associated with an NO/cGMP-dependent protein kinase network by administering to a mammalian subject an effective amount of a compound that modulates the expression of a polynucleotide selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104. or the activity or expression of a polypeptide encoded by a polynucleotide selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104.

Methods of treating an individual are intended to include preventing, ameliorating, curing, and reducing the severity of a disease or symptoms associated with a disease. Those skilled in the art understand that any degree of reduction in severity of ADHD, hypertension or other disease associated with a NO/cGMP-dependent kinase network in a mammal can improve the health or quality of life of an individual. The effect of the therapy can be determined by those skilled in the art, by comparison to baseline values for symptoms or clinical or diagnostic markers associated with the disorder.

A compound identified by the methods of the invention can be administered to a mammal for the purpose of treating ADHD, hypertension or other disease associated with a NO/cGMP-dependent kinase network in a mammal. An effective amount of a compound to treat ADHD, hypertension or other disease associated with a NO/cGMP-dependent kinase network in a mammal is an amount of the compound required to effect a decrease in a symptom or severity of ADHD, hypertension or other disease associated with a NO/cGMP-dependent kinase network in a mammal respectively. The dosage required to be therapeutically effective will depend, for example, on the particular disease, the route and form of administration, the weight and condition of the individual, and previous or concurrent therapies. The appropriate amount considered to be an effective dose for a particular application of the method can be determined by those skilled in the art, using the guidance provided herein. For example, the amount can be determined from diagnostic or gene expression assays described herein. One skilled in the art will recognize that the condition of the patient can be monitored throughout the course of therapy and that the amount of compound that is administered can be adjusted accordingly.

An effective amount can be, for example, between about 10 μg/kg to 500 mg/kg body weight, for example, between about 0.1 mg/kg to 100 mg/kg, or preferably between about 1 mg/kg to 50 mg/kg, depending on the treatment regimen. For example, if a compound that modulates ADHD, hypertension or other disease associated with a NO/cGMP-dependent kinase network in a mammal is administered from one to several times a day, then a lower dose would be needed than if a formulation were administered weekly, or monthly or less frequently. Similarly, formulations that allow for timed-release of such a compound would provide for the continuous release of a smaller dose than would be administered as a single bolus dose. For example, a compound that modulates ADHD, hypertension or other disease associated with a NO/cGMP-dependent kinase network in a mammal can be administered at between about 1-5 mg/kg/week.

A compound that modulates ADHD, hypertension or other disease associated with a NO/cGMP-dependent kinase network in a mammal can be delivered systemically, such as intravenously or intra arterially. Such a compound can also be administered locally at a site of the pathological condition. Appropriate sites for administration of a compound that modulates ADHD, hypertension or other disease associated with a NO/cGMP-dependent kinase network in a mammal are known, or can be determined, by those skilled in the art depending on the clinical indications of the individual being treated. For example, a compound can be administered in the brain or nervous system of an individual having ADHD or in the vasculature of an individual having hypertension. A compound can be provided in a substantially purified form in pharmaceutically acceptable formulations using formulation methods known to those of ordinary skill in the art. These formulations can be administered by standard routes, including for example, topical, transdermal, intra peritoneal, intra cranial, intracerebroventricular, intra cerebral, intra vaginal, intrauterine, oral, rectal or parenteral (e.g., intravenous, intra spinal, subcutaneous or intramuscular) routes. Methods for such routes of administration are well known to those skilled in the art.

In addition, a compound that modulates ADHD, hypertension or other disease associated with a NO/cGMP-dependent kinase network in a mammal can be incorporated into biodegradable polymers allowing for sustained release of the compound, the polymers being implanted in the vicinity of where drug delivery is desired, for example, in the brain for an ADHD patient or in a vascular tissue for a hypertension patient. Osmotic minipumps also can be used to provide controlled delivery of specific concentrations of such compounds and formulations through cannulae to the site of interest, such as directly into a nervous or vascular tissue. The biodegradable polymers and their use are described, for example, in detail in Brem et al., J. Neurosurg. 74:441-446 (1991).

A compound that modulates ADHD, hypertension or other disease associated with a NO/cGMP-dependent kinase network in a mammal can be administered as a solution or suspension together with a pharmaceutically acceptable medium in such a manner to ensure proper distribution in vivo. For example, the blood-brain barrier excludes many highly hydrophilic compounds. To ensure that the compounds intended to treat ADHD cross the blood brain barrier, they can be formulated, for example, in liposomes, or chemically derivatized. Other pharmaceutically acceptable media include, for example, water, sodium phosphate buffer, phosphate buffered saline, normal saline or Ringer's solution or other physiologically buffered saline, or other solvent or vehicle such as a glycol, glycerol, an oil such as olive oil or an injectable organic ester so long as the formulation is of sufficient purity and quality for use in humans, sterile and substantially free from contaminating particles and organisms.

A pharmaceutically acceptable medium can additionally contain physiologically acceptable compounds that act, for example, to stabilize the compound that modulates ADHD, hypertension or other disease associated with a NO/cGMP-dependent kinase network in a mammal. Such physiologically acceptable compounds include, for example, carbohydrates such as glucose, sucrose or dextrans; antioxidants such as ascorbic acid or glutathione; chelating agents such as EDTA, which disrupts microbial membranes; divalent metal ions such as calcium or magnesium; low molecular weight proteins; lipids or liposomes; or other stabilizers or excipients.

A compound that modulates ADHD, hypertension or other disease associated with a NO/cGMP-dependent kinase network in a mammal can be administered in conjunction with other therapies. For example, for treating ADHD, the compound can be administered prior to, during, or subsequent to other ADHD treatments including, for example, behavior modification or drug therapies such as methylphenidate (ritalin), amphetamines, pemoline or cylert. Similarly, for treating hypertension the compound can be administered prior to, during, or subsequent to treatments, such as controlled regimens of diet or exercise or drug therapies such as diuretics, ACE inhibitors, beta blockers, vasodilators or calcium channel blockers. For a description of treatments for ADHD or hypertension see, for example, The Merck Manual, Sixteenth Ed, (Berkow, R., Editor) Rahway, N.J., 1992.

It will be understood that the efficacy and safety of a compound in laboratory mammals can be evaluated before administering the compound to humans or veterinary animals. For example, the compound can be tested for its maximal efficacy and any potential side-effects using several different invertebrates or laboratory mammals, across a range of doses, in a range of formulations, and at various times of day, such as before or after sleeping, before or after eating, and the like. Generally, a compound identified using the methods of the invention will cause few or no deleterious or unwanted side effects.

Thus, in one embodiment, the invention provides a method of determining the efficacy of a compound in treating ADHD, hypertension or other disease associated with a NO/cGMP-dependent kinase network in a mammal. The method consists of administering a compound to an individual having ADHD, hypertension or other disease associated with a NO/cGMP-dependent kinase network in a mammal, and determining an effect of the compound on the expression profile of the individual. A compound that modulates the expression profile of the individual to correspond to an unaffected or normal profile indicates that the compound is effective in treating ADHD, hypertension or other disease associated with a NO/cGMP-dependent kinase network in a mammal.

Once genes associated with ADHD, hypertension or other disease associated with a NO/cGMP-dependent kinase network in a mammal have been identified, the expression or activity of such genes in humans or other mammals can be selectively targeted in order to prevent or treat the disease. The diagnostic, screening and validation methods of the invention are useful in determining appropriate genes to target and appropriate therapeutic compounds to use for a particular indication.

If desired, treating an individual having ADHD, hypertension or other disease associated with a NO/cGMP-dependent kinase network in a mammal can be achieved with gene therapy. Methods of introducing and expressing polynucleotides in animals, including humans, are well known in the art. For example, gene therapy methods can be performed by ex vivo methods, wherein cells (e.g. hematopoietic cells, including stem cells) are removed from the body, engineered to express a polypeptide associated with invertebrate foraging or with modulation of ADHD, hypertension or other disease associated with a NO/cGMP-dependent kinase network in a mammal, and returned to the body. Gene therapy methods can also be performed by in situ methods, such that an expressible polynucleotide molecule is placed directly into an appropriate tissue, such as the brain or CNS, by a direct route such as injection or implantation during surgery. Gene therapy methods can also be performed in vivo, wherein the expressible polynucleotide molecule is administered systemically, such as intravenously. Appropriate vectors for gene therapy can be determined by those skilled in the art for a particular application of the method, and include, but are not limited to, retro viral vectors (e.g. replication-defective MuLV, HTLV, and HIV vectors); adeno viral vectors; adeno-associated viral vectors; herpes simplex viral vectors; and non-viral vectors. Appropriate formulations for delivery of polynucleotides can also be determined by those skilled in the art, and include, for example, liposomes; polycationic agents; naked DNA; and DNA associated with or conjugated to targeting molecules (e.g. antibodies, ligands, lectins, fusogenic peptides, or HIV tat peptide). Gene therapy methods, including considerations for choice of appropriate vectors, promoters, formulations and routes of delivery, are reviewed, for example, in Anderson, Nature 392:25-30 (1998).

It is understood that modifications which do not substantially affect the activity of the various embodiments of this invention are also provided within the definition of the invention provided herein. Accordingly, the following examples are intended to illustrate but not limit the present invention.

### EXAMPLE I

Measuring Foraging in for^{R} and for^{S} Strains of Drosophila

This example shows the measurement of foraging scores for strains of Drosophila that have varying foraging behaviors.

Drosophila lines with various alleles of the foraging locus (for), encoding one of two forms of cGMP-dependent protein kinase were used. The for^{R} strain, termed Rover, is a naturally occurring allelic variant which demonstrates high activity in the presence of food or in the fed state. The for^{S} strain, termed sitter, is also a naturally occurring variant and shows low activity in the presence of food or in a fed state. The naturally occurring for^{R} and for^{S} strains were obtained from M. Sokolowski (York University, North York, Ontario, Canada) and have been described previously, for example, in de Belle et al., Heredity 59:73 (1987). In addition, a variant demonstrating reduced activity in the presence of food, termed for^{s2}, was used. The for^{s2} variant was produced by X-ray induced mutation at the for locus and does not complement the fo^{R} or for^{S} alleles as described, for example, in de Belle et al. Genetics 123:157 (1989)

These strains were tested in a foraging maze constructed according to the design of Hirsch (J. Comp. Physiol. Psychol. 52:304-308 (1959)) as modified by McMillan and McGuire (Behav. Genet. 22:557-573 (1992)), shown in FIG. 1.

The foraging maze was fashioned from plexiglass and the maze chambers were hollowed out as semi-circular depressions in the slab, such that when the two slabs were bolted together a circular tube was formed in maze. This design thus made it possible to observe the entire time course of the assay. The maze is placed flat on a uniform light source so that each path from the start tube to a collection tube is equal distant and not subject to an elevation gain and the maze is evenly illuminated throughout.

For each strain, a foraging score was determined. In separate measurements, twenty to thirty flies, 3-5 days old were starved overnight, allowed to briefly (15 minutes) feed on 0.25 M sucrose, then placed into the start tube of the maze. After 5 minutes, the number of flies that had reached any of the final collection tubes was summed and expressed as a percentage of the total number tested to yield the “foraging score.” Standard errors were also calculated (SEM). The foraging measurements were repeated 4-5 times (N) for each strain (see Table 2).

TABLE 2

| | N (20-30 flies | | |
|---|---|---|---|
| Strain | in each) | Foraging score | SEM |
| for^{R} | 4 | 76.1 | 10.3 |
| for^{s} | 5 | 36.7 | 14.8 |
| for^{s2} | 4 | 30.9 | 11.2 |

As shown in Table 2 the for^{R} strain showed a roughly 2 fold increase in foraging score as compared to the for^{S} strain and a roughly 2.5 fold increase in foraging score as compared to the for^{s2} strain.

These results demonstrate an assay useful in quantitatively determining a foraging behavior for a group of flies. These results further demonstrate statistically significant differences in foraging behaviors for Rovers and sitters.

### EXAMPLE II

Molecular Components of the NO/cGMP-Dependent Protein Kinase as Identified in Drosophila

This example demonstrates the identification of polynucleotides and polypeptides that are differently expressed in Drosophila having a for^{R} allele compared to Drosophila that are homozygous for the for^{s2} allele.

The genetic differences between for^{R} and for^{s2} were used as a basis for identifying genes in the NO/cGMP-dependent protein kinase network based on their differential expression between these two strains. Differential display of mRNA was used to assay differences in mRNA levels and 2 dimensional polyacrylamide gel electrophoresis in tandem with mass spectrometry was used to identify differences in protein expression levels.

For each strain, twenty flies, 3-5 days old were starved overnight then allowed to feed on 0.25 M sucrose for 15 minutes prior to decapitation. For mRNA differential display, 20 fly heads were homogenized in TRIzol (life Technologies, Inc. Frederick, Md.) and extracted according the manufacturer's instructions. Differential display of mRNA was performed as described in Cirelli et al., supra. Sequences were analyzed using the BLAST program at either the Berkeley Drosophila Genome Project or NCBI websites.

For protein analysis, 35 fly heads were homogenized and separated by 2 dimensional polyacrylamide gel electrophoresis using the methods described in Unlu et al., Electrophoresis 18:2071 (1997). Spots were matched between gels and those showing the strongest differences were then excised with a scalpel and subjected to in gel trypsin digestion, extraction, purification and then analyzed by MALDI-TOF as described in Helman et al. Anal. Biochem. 224:451 (1995). The resulting spectra were analyzed by matching peptide patterns to those in the Profound database administered by the Rockefeller University.

A number of mRNA sequences that were differentially expressed could be identified as expressed sequence tags (EST's) in the Berkeley Drosophila Genome Project or NCBI databases and are provided as SEQ ID NOS: 1-46. In addition a number of previously cloned Drosophila genes were identified as shown in Table 3.

TABLE 3

| Change in for^{R} vs. for^{s2} | differentially expressed mRNA |
|---|---|
| increase | DEAD-box protein (Ddx1) |
| increase | CNS-specific protein Noe (noe) |
| increase | 14-3-3 ε |
| increase | cellular repressor of E1A- |
| | stimulated genes (CREG) |
| increase | casein kinase II α subunit |
| increase | mRNA sequence similar to |
| | syntaxin I |
| increase | ADP/ATP translocase |
| increase | mitochondrial porin |
| decrease | neuron specific zinc finger |
| | transcription factor (scratch) |
| decrease | ectozoan-regulated (E93) |
| decrease | centrosomal and chromosomal |
| | factor (ccf) |

A number of proteins that were differentially expressed could be identified as shown in Table 4.

TABLE 4

| Change in for^{R} vs. for^{s2} | differentially expressed |
|---|---|
| increase | cellular repressor of E1A- |
| | stimulated genes (CREG) |
| increase | activin β precursor |
| increase | dynamic-like (S17974) |
| increase | paramyosin |
| increase | mitochondrial ATP synthase α |
| | subunit |
| decrease | Fas-associated factor (FFAF) |
| decrease | lamin precursor |
| decrease | alcohol dehydrogenase (Adh) |

Additionally, a number of proteins were identified according to mass and isoelectric point (pI) as shown in table 5.

TABLE 5

| | molecular weight | |
|---|---|---|
| sample number | (kD) | pI |
| 1 | 50 | 4.1 |
| 4 | 28 | 8.7 |
| 8 | 36 | 6.0 |
| 9 | 34 | 6.3 |
| 11 | 25 | 5.9 |
| 13 | 12 | 5.7 |
| 14 | 12 | 6.4 |
| 18 | 29 | 6.5 |

The mRNA sequences and polypeptides identified in this example represent targets for modulation of ADHD, hypertension or other diseases associated with a NO/cGMP-dependent protein kinase network in a mammal. In addition the mRNA and polypeptides identified in this example can be used as a molecular fingerprint or diagnostic of ADHD, hypertension or other diseases associated with a NO/cGMP-dependent protein kinase network in a mammal. Because differences can be observed between naturally tolerable phenotypes (i.e. changes from Rover to sitter behavior) with no loss of fitness or health this fingerprint can be used to determine the extent to which a test compound will modulate foraging and therefore ADHD, hypertension or other diseases associated with a NO/cGMP-dependent protein kinase network in a mammal.

### EXAMPLE III

Effects of an Alcohol Dehydrogenase Allele Adhⁿ¹ on Foraging

This example demonstrates use of a foraging assay to determine the effects of various alleles, independent of or in combination with Rover/sitter, on the activity of Drosophila in response to feeding and starvation.

The Adhⁿ¹ allele of the alcohol dehydrogenase locus results in 20% of the normal level of alcohol dehydrogenase activity as described in Chenevert et al., Biochem. J. 308:419 (1995). Adhⁿ¹ flies were obtained from The Drosophila Stock Center, Dept. of Biology, University of Indiana, Bloomington Ind. Doubly homozygous mutants were made with Rover or sitter and a mutant of the ipp locus (ipp²) as described in Greenspan, supra. A mutant of another component of the inositol phosphate signaling pathway, the inositol 1,4,5-tri-phosphate receptor (Itp-r83A; Acharya et al. Neuron 18:881 (1997)) was also tested for its activity on foraging. The ipp² and Itp-r83A flies were obtained from C. Zuker, Dept of Biology, University of California, San Diego.

All strains were assayed for foraging behavior as described in Example I. Foraging scores are presented in Table 6.

TABLE 6

| Strain | Fed | starved | starved then fed |
|---|---|---|---|
| for^{R} | 82.4 | 54.8 | 76.1 |
| for^{s2} | 48.0 | 39.2 | 30.9 |
| Adhⁿ¹ | 8.9 | 15.0 | 31.1 |
| for^{R}; ipp² | 100.0 | 70.8 | 45.5 |
| for^{s2}; ipp² | 40.0 | 33.3 | 38.2 |
| Itp-r83A¹ | 8.9 | 23.3 | 6.7 |

As shown in Table 6, While less active than Rover or Sitter under normal or starved conditions, Adhⁿ¹ produces a Rover-like increase in its foraging in response to starvation followed by brief feeding. This is consistent with what would be expected based on the reduction in And levels seen in Rovers vs. sitters in Example II.

Also shown in Table 6, doubly homozygous mutants of for^{R};ipp² showed a dramatic alteration of Rover phenotype after being starved then fed when compared to for^{R} flies. However, very little effect was observed on the sitter phenotype for doubly homozygous for^{s2}; ipp² when compared to for^{s2} flies under any condition. This specific interaction with Rover identifies ipp as a target for modulating foraging behavior in invertebrates and therefore ADHD, hypertension or a disease associated with a NO/cGMP-dependent protein kinase in a mammal.

As further shown in Table 6, a mutant of the inositol phosphate signaling pathway, the inositol 1,4,5-tri-phosphate receptor (Itp-r83A) also shows an influence on foraging, exhibiting a phenotype opposite that of Rover. More specifically, Itp-r83A flies displayed more activity under conditions of starvation than under either of the other conditions. This identifies Itp-r83A as a target for modulating foraging behavior in invertebrates and therefore ADHD, hypertension or a disease associated with a NO/cGMP-dependent protein kinase in a mammal.

### EXAMPLE IV

Changes in Foraging Behavior Induced by Ritalin

This Example demonstrates biological activity of methylphenidate (ritalin) in Drosophila Melanogaster and its effects on sleep were evaluated

Methyiphenidate is a biologically active compound in mammals that is used clinically to ameliorate symptoms associated with attention-def icit/hyperactivity disorder (ADHD). Methylphenidate can also be used to treat excessive daytime sleepiness associated with many sleep disorders including narcolepsy. The therapeutic role of this medication has proven effective not only for treating excessive sleepiness but also for improving associated deficits in both affect and cognition. In order to determine whether methylphenidate is biologically active in an invertebrate, methylphenidate was administered to Drosophila melanogaster and its effects on sleep were evaluated.

Wild-type Canton-S (CS) flies were placed into a 50 ml vial containing yeast, dark corn syrup and agar food. Each colony was housed at 25° C. with a relative humidity of 50% in a Forma Scientific incubator. The flies were maintained on a 12:12 light/dark cycle with “lights-on” commencing at 8:00 am and “lights-off” commencing at 8:00 pm to yield darkness. Virgin females were collected and maintained in 50 ml vials for one day. During the second day, flies were individually placed into glass tubes (65 mm in length, 5 mm I.D.) containing about lomm of fresh food and the tubes were placed into the Drosophila Activity Monitoring System (Trikinetics, Waltham, Mass.). The flies remained undisturbed for the following 48 hours. The amount and distribution of rest was evaluated during the third day to ensure that the flies had adapted to the apparatus as previously described in Shaw et. al., Science 287:1834-1837 (2000). On day 4, two hours before lights-off, flies were transferred to fresh glass tubes containing only standard laboratory food or to tubes with 0.5 mg/ml methylphenidate that had been dissolved in the food. The amount and distribution of rest during the following 14 hours was then evaluated.

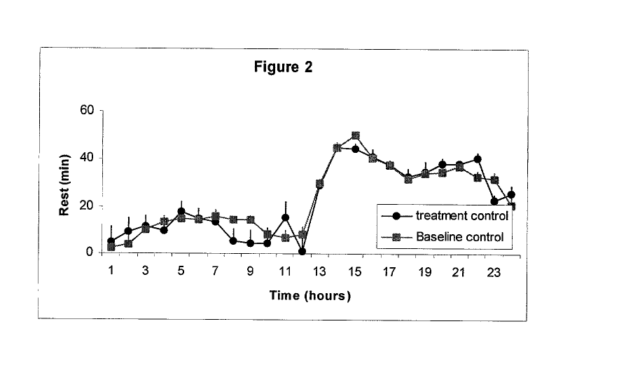

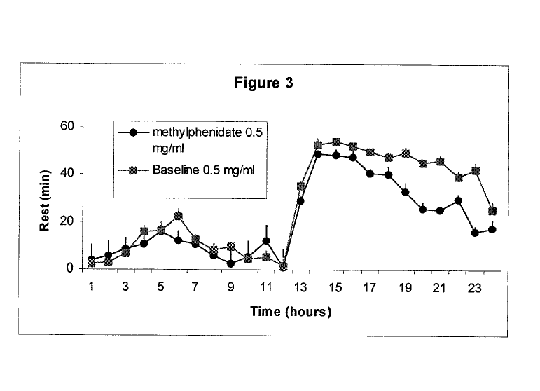

The amount and distribution of sleep during baseline was similar to previously published results in all treatment groups Shaw et. al., Science 287:1834-1837 (2000). Flies that were transferred to normal food two hours before the beginning of the main rest period did not show any reduction in sleep over the course of the 12 hour dark period when compared to flies that remained in the original tube as shown in FIG. 2. The similarity in rest pattern for both baseline and transferred flies indicates that the transfer process did not alter the sleep-wake cycle. In contrast, flies that were transferred to tubes containing 0.5 mg/ml methylphenidate showed a significant and substantial decrease in rest that increased throughout the rest period (p<0.05) as shown in FIG. 3.

These results demonstrate that methylphenidate is biologically active in invertebrates, increasing waking and reducing sleep during the normal sleep period in the fruit fly. Methylphenidate has an effect in treating excessive daytime sleepiness associated with narcolepsy in humans. Furthermore, the administration of methylphenidate to normal subjects during their typical sleep times indicates that it can effectively used to reduce sleep drive. In addition, methylphenidate can alleviate the decline in cognitive functioning associated with decreased levels of arousal induced by chronic sleep deprivation. Therefore, these results demonstrate that methylphenidate has similar biological effect in both mammals and invertebrates.

Throughout this application various publications have been referenced. The disclosure of these publications in their entireties are hereby incorporated by reference in this application in order to more fully describe the state of the art to which this invention pertains.

Although the invention has been described with reference to the disclosed embodiments, those skilled in the art will readily appreciate that the specific details are only illustrative of the invention. It is understood that modifications which do not substantially affect the activity of the various embodiments of this invention are also included within the definition of the invention provided herein. Therefore, it should be understood that various modifications can be made without departing from the spirit of the invention. Accordingly, the invention is limited only by the following claims.

We claim:

1. A method of identifying a compound that modulates ADHD in a mammal, comprising:
(a) administering a test compound to an invertebrate; and
(b) measuring a foraging behavior of said invertebrate, wherein a compound that modulates the foraging behavior of said invertebrate is characterized as a compound that modulates ADHD in a mammal.

2. The method of claim 1, wherein said compound reduces ADHD in a mammal.

3. The method of claim 1, wherein said foraging behavior comprises a phenotype of the for gene.

4. The method of claim 1, wherein said compound increases the foraging behavior of a sitter.

5. The method of claim 4, wherein said compound reduces ADHD in a mammal.

6. The method of claim 1, wherein said compound decreases the foraging behavior of a Rover.

7. The method of claim 6, wherein said compound reduces ADHD in a mammal.

8. The method of claim 1, wherein said compound modulates the expression or activity of one or more mammalian polypeptides.

9. The method of claim 1, wherein said invertebrate is an insect.

10. The method of claim 1, wherein said invertebrate is Drosophila melanogaster.

11. The method of claim 10, wherein said Drosophila melanogaster is an adult.

12. The method of claim 10, wherein said Drosophila melanogaster is a larva.

13. The method of claim 1, wherein said mammal is human.

14. A method of identifying a compound that modulates hypertension in a mammal, comprising:
(a) administering a test compound to an invertebrate; and
(b) measuring a foraging behavior of said invertebrate, wherein a compound that modulates the foraging behavior of said invertebrate is characterized as a compound that modulates hypertension in a mammal.

15. The method of claim 14, wherein said compound reduces hypertension in a mammal.

16. The method of claim 14, wherein said compound increases the foraging behavior of a sitter.

17. The method of claim 16, wherein said compound reduces hypertension in a mammal.

18. The method of claim 14, wherein said foraging behavior comprises a phenotype of the for gene.

19. The method of claim 14, wherein said compound decreases the foraging behavior of a Rover.

20. The method of claim 19, wherein said compound reduces hypertension in a mammal.

21. The method of claim 14, wherein said compound modulates the expression or activity of one or more mammalian polypeptides.

22. The method of claim 14, wherein said invertebrate is an insect.

23. The method of claim 14, wherein said invertebrate is Drosophila melanogaster.

24. The method of claim 23, wherein said Drosophila melanogaster is an adult.

25. The method of claim 23, wherein said Drosophila melanogaster is a larva.

26. The method of claim 14, wherein said mammal is human.

27. A method of identifying a compound that modulates ADHD in a mammal, comprising:
(a) administering a test compound to an invertebrate;
(b) measuring an expression level for one or more polynucleotides in said invertebrate; and
(c) comparing said expression level for one or more polynucleotides in said invertebrate to an expression level of one or more polynucleotides in a reference invertebrate, a compound having the effect of modulating said expression level of one or more polynucleotides associated with invertebrate foraging behavior in said test invertebrate relative to said reference invertebrate is identified as a compound that modulates ADHD in a mammal.

28. The method of claim 27, wherein said compound reduces ADHD in a mammal.

29. The method of claim 27, wherein said foraging behavior comprises a phenotype of the for gene.

30. The method of claim 27, wherein said compound increases the foraging behavior of a sitter.

31. The method of claim 30, wherein said compound decreases severity of ADHD in a mammal.

32. The method of claim 27, wherein said compound decreases the foraging behavior of a Rover.

33. The method of claim 32, wherein said compound decreases severity of ADHD in a mammal.

34. The method of claim 27, wherein a compound that has the effect of increasing expression of one or more polynucleotides in said invertebrate relative to said reference invertebrate is identified as a compound that decreases severity of ADHD.

35. The method of claim 27, wherein a compound that has the effect of decreasing expression of a specific polynucleotide in said invertebrate relative to said reference invertebrate is identified as a compound that decreases a symptom of ADHD.

36. The method of claim 27, further comprising step (d) measuring an expression level for one or more polynucleotide in said reference invertebrate, wherein said test compound is administered to said reference invertebrate.

37. The method of claim 27, wherein said invertebrate exhibits substantially the same foraging behavior as said reference invertebrate before said compound is administered.

38. The method of claim 27, wherein said invertebrate exhibits a different foraging behavior from said reference invertebrate before said compound is administered.

39. The method of claim 27, wherein said invertebrate exhibits substantially the same foraging behavior as said reference invertebrate after said compound is administered.

40. The method of claim 27, wherein said invertebrate exhibits a different foraging behavior from said reference invertebrate after said compound is administered.

41. The method of claim 27, wherein said invertebrate is an insect.

42. The method of claim 27, wherein said invertebrate is Drosophila melanogaster.

43. The method of claim 42, wherein said Drosophila melanogaster is an adult.

44. The method of claim 42, wherein said Drosophila melanogaster is a larva.

45. The method of claim 27, wherein said mammal is human.

46. The method of claim 27, wherein said one or more polynucleotides having modulated expression levels are selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104.

47. The method of claim 46, wherein a mammalian polynucleotide comprises a nucleic acid sequence substantially the same as said one or more polynucleotides comprising said sequence selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104.

48. A method of identifying a compound that modulates hypertension in a mammal, comprising:
(a) administering a test compound to an invertebrate;
(b) measuring an expression level of one or more polynucleotides in said invertebrate; and
(c) comparing said expression level of one or more polynucleotides in said invertebrate to an expression level of one or more polynucleotides in a reference invertebrate, a compound having the effect of modulating said expression level of one or more polynucleotides associated with invertebrate foraging behavior in said test invertebrate relative to said reference invertebrate is identified as a compound that modulates hypertension in a mammal.

49. The method of claim 48, wherein said compound reduces hypertension in a mammal.

50. The method of claim 48, wherein said foraging behavior comprises a phenotype of the for gene.

51. The method of claim 48, wherein said compound increases the foraging behavior of a sitter.

52. The method of claim 48, wherein said compound decreases severity of hypertension in a mammal.

53. The method of claim 48, wherein said compound decreases the foraging behavior of a Rover.

54. The method of claim 53, wherein said compound decreases severity of hypertension in a mammal.

55. The method of claim 48, wherein a compound that has the effect of increasing expression of one or more polynucleotides in said invertebrate relative to said reference invertebrate is identified as a compound that decreases severity of ADHD.

56. The method of claim 48, wherein a compound that has the effect of decreasing expression of a specific polynucleotide in said test invertebrate relative to said reference invertebrate is characterized as a compound that decreases a symptom of ADHD.

57. The method of claim 48, further comprising step (d) measuring an expression level for one or more polynucleotide in said reference invertebrate, wherein said test compound is administered to said reference invertebrate.

58. The method of claim 48, wherein said invertebrate exhibits substantially the same foraging behavior as said reference invertebrate before said compound is administered.

59. The method of claim 48, wherein said invertebrate exhibits a different foraging behavior from said reference invertebrate before said compound is administered.

60. The method of claim 48, wherein said invertebrate exhibits substantially the same foraging behavior as said reference invertebrate after said compound is administered.

61. The method of claim 48, wherein said invertebrate exhibits a different foraging behavior from said reference invertebrate after said compound is administered.

62. The method of claim 48, wherein said invertebrate is an insect.

63. The method of claim 48, wherein said invertebrate is Drosophila melanogaster.

64. The method of claim 63, wherein said Drosophila melanogaster is an adult.

65. The method of claim 63, wherein said Drosophila melanogaster is a larva.

66. The method of claim 48, wherein said mammal is human.

67. The method of claim 48, wherein said one or more polynucleotides having modulated expression levels are selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104.

68. The method of claim 67, wherein a mammalian polynucleotide comprises a nucleic acid sequence substantially the same as said one or more polynucleotides comprising said sequence selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104.

69. A method of identifying a polynucleotide that correlates with ADHD in a mammal, comprising:
(a) obtaining a first and a second strain of an invertebrate;
(b) subjecting said first and second invertebrate strains to conditions in which said first strain exhibits a foraging behavior different than a foraging behavior exhibited by said second strain;
(c) measuring an expression level for one or more polynucleotides in said first and second strains, and
(d) identifying one or more polynucleotides that are differentially expressed in said first strain relative to said second strain, wherein a mammalian polynucleotide comprising substantially the same nucleic acid sequence as said one or more differentially expressed polynucleotides correlates with ADHD in a mammal.

70. The method of claim 69, wherein increased expression of said mammalian polynucleotide correlates with an increase or decrease in severity of ADHD.

71. The method of claim 69, wherein decreased expression of said mammalian polynucleotide correlates with an increase or decrease in severity of ADHD.

72. The method of claim 69, wherein said foraging behavior comprises a phenotype of the for gene.

73. The method of claim 69, wherein said different behavior exhibited by said first strain is reduced foraging behavior in a Rover.

74. The method of claim 69, wherein said different behavior exhibited by said first strain is increased foraging behavior in a sitter.

75. The method of claim 69, wherein said mammalian polynucleotide comprises a nucleic acid sequence substantially the same as a nucleic acid sequence selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104.

76. The method of claim 69, wherein said mammalian polynucleotide encodes a polypeptide having characteristics selected from the group consisting of molecular weight of 50 kD and pI of 4.1, a molecular weight of 28 kD and pI of 8.7, a molecular weight of 36 kD and pI of 6.0, a molecular weight of 34 kD and pI of 6.3, a molecular weight of 25 kD and pI of 5.9, a molecular weight of 12 kD and pI of 5.7, a molecular weight of 12 kD and pI of 6.4, a molecular weight of 12 kD and pI of 6.4, or a molecular weight of 29 kD and pI of 6.5.

77. The method of claim 69, wherein said one or more polynucleotides that are differentially expressed in said first strain relative to said second strain have increased expression and said mammalian polynucleotide has increased expression when involved in ADHD in a mammal.

78. The method of claim 69, wherein said one or more polynucleotide that are differentially expressed in said first strain relative to said second strain have decreased expression and said mammalian polynucleotide has decreased expression when involved in ADHD in a mammal.

79. A method of identifying a polynucleotide that correlates with hypertension comprising:
(a) obtaining a first and a second strain of an invertebrate;
(b) subjecting said first and second invertebrate strains to conditions in which said first strain exhibits a foraging behavior different than a foraging behavior exhibited by said second strain;
(c) measuring an expression level for one or more polynucleotide in said first and second strains, and
(d) identifying one or more polynucleotides that are differentially expressed in said first strain relative to said second strain, wherein a mammalian polynucleotide comprising substantially the same nucleic acid sequence as said one or more differentially expressed polynucleotides correlates with hypertension.

80. The method of claim 79, wherein increased expression of said mammalian polynucleotide correlates with an increase or decrease in severity of hypertension.

81. The method of claim 79, wherein decreased expression of said mammalian polynucleotide correlates with an increase or decrease in severity of hypertension.

82. The method of claim 79, wherein said foraging behavior comprises a phenotype of the for gene.

83. The method of claim 79, wherein said different behavior exhibited by said first strain is reduced foraging behavior in a Rover.

84. The method of claim 79, wherein said different behavior exhibited by said first strain is increased foraging behavior in a sitter.

85. The method of claim 79, wherein said mammalian polynucleotide comprises a nucleic acid sequence substantially the same as a nucleic acid sequence selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104.

86. The method of claim 79, wherein said mammalian polynucleotide encodes a polypeptide having the characteristics selected from the group consisting of molecular weight of 50 kD and pI of 4.1, a molecular weight of 28 kD and pI of 8.7, a molecular weight of 36 kD and pI of 6.0, a molecular weight of 34 kD and pI of 6.3, a molecular weight of 25 kD and pI of 5.9, a molecular weight of 12 kD and pI of 5.7, a molecular weight of 12 kD and pI of 6.4, a molecular weight of 12 kD and pI of 6.4, and a molecular weight of 29 kD and pI of 6.5.

87. The method of claim 79, wherein said one or more polynucleotides that are differentially expressed in said first strain relative to said second strain have increased expression and said mammalian polynucleotide has increased expression when involved in hypertension in a mammal.

88. The method of claim 79, wherein said one or more polynucleotide that are differentially expressed in said first strain relative to said second strain have decreased expression and said mammalian polynucleotide has decreased expression when involved in hypertension in a mammal.

89. An isolated polynucleotide having ADHD-altering activity in a mammal, or fragment thereof, comprising substantially the same nucleic acid sequence as a polynucleotide selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104.

90. An isolated polynucleotide having hypertension-altering activity in a mammal, or fragment thereof, comprising substantially the same nucleic acid sequence as a polynucleotide selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104.

91. An isolated polypeptide having ADHD-altering activity in a mammal, or fragment thereof, comprising substantially the same amino acid sequence as an amino acid sequence encoded by a nucleic acid sequence selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104.

92. An antibody specific for the isolated polypeptide of claim 91.

93. The antibody of claim 92, wherein said antibody is a monoclonal antibody.

94. The antibody of claim 92, wherein said antibody is a polyclonal antibody.

95. An isolated polypeptide having hypertension-altering activity in a mammal, or fragment thereof, comprising substantially the same amino acid sequence as an amino acid sequence encoded by a nucleic acid sequence selected from the group consisting of SEQ ID NOS: 1-75, SEQ ID NO: 77, SEQ ID NO: 79, SEQ ID NO: 85, SEQ ID NO: 87, SEQ ID NO: 89, SEQ ID NO: 91, SEQ ID NO: 93, SEQ ID NO: 95, SEQ ID NO: 100, SEQ ID NO: 102 and SEQ ID NO: 104.

96. An isolated polypeptide having ADHD-altering activity in a mammal, or fragment thereof, comprising a polypeptide having the characteristics selected from the group consisting of molecular weight of 50 kD and pI of 4.1, a molecular weight of 28 kD and pI of 8.7, a molecular weight of 36 kD and pI of 6.0, a molecular weight of 34 kD and pI of 6.3, a molecular weight of 25 kD and pI of 5.9, a molecular weight of 12 kD and pI of 5.7, a molecular weight of 12 kD and pI of 6.4, a molecular weight of 12 kD and pI of 6.4, and a molecular weight of 29 kD and pI of 6.5.

100. An isolated polypeptide having hypertension-altering activity in a mammal, or fragment thereof, comprising a polypeptide having the characteristics selected from the group consisting of molecular weight of 50 kD and pI of 4.1, a molecular weight of 28 kD and pI of 8.7, a molecular weight of 36 kD and pI of 6.0, a molecular weight of 34 kD and pI of 6.3, a molecular weight of 25 kD and pI of 5.9, a molecular weight of 12 kD and pI of 5.7, a molecular weight of 12 kD and pI of 6.4, a molecular weight of 12 kD and pI of 6.4, and a molecular weight of 29 kD and pI of 6.5.
